SEQUENCE LISTING (10 sequences)

SEQ ID NO: 1 - DNA, 24 bp, synthetic construct
  misc_feature 1..24: note = Description of sequence: Primer, name: CD16 Forward Primer
  source 1..24: mol_type = other DNA
caaatccttcatcatgtcagttcc

SEQ ID NO: 2 - DNA, 22 bp, synthetic construct
  misc_feature 1..22: note = Description of sequence: Primer, name: CD16 Reverse Primer
  source 1..22: mol_type = other DNA
gaaccaagaaatgttgcgctta

SEQ ID NO: 3 - DNA, 21 bp, synthetic construct
  misc_feature 1..21: note = Description of sequence: Primer, name: CD56 Forward Primer
  source 1..21: mol_type = other DNA
gcagatgggagaggatggaaa

SEQ ID NO: 4 - DNA, 21 bp, synthetic construct
  misc_feature 1..21: note = Description of sequence: Primer, name: CD56 Reverse Primer
  source 1..21: mol_type = other DNA
tctggtttccactcggaggag

SEQ ID NO: 5 - DNA, 23 bp, synthetic construct
  misc_feature 1..23: note = Description of sequence: Primer, name: NKG2D Forward Primer
  source 1..23: mol_type = other DNA
tcaagatcttccctctctgagca

SEQ ID NO: 6 - DNA, 21 bp, synthetic construct
  misc_feature 1..21: note = Description of sequence: Primer, name: NKG2D Reverse Primer
  source 1..21: mol_type = other DNA
cgaatccaccccatcaaatac

SEQ ID NO: 7 - DNA, 21 bp, synthetic construct
  misc_feature 1..21: note = Description of sequence: Primer, name: NKp46 Forward Primer
  source 1..21: mol_type = other DNA
actaggccggcagaatctgag

SEQ ID NO: 8 - DNA, 21 bp, synthetic construct
  misc_feature 1..21: note = Description of sequence: Primer, name: NKp46 Reverse Primer
  source 1..21: mol_type = other DNA
cgagagggtgggtgtgtcata

SEQ ID NO: 9 - DNA, 21 bp, synthetic construct
  misc_feature 1..21: note = Description of sequence: Primer, name: CD107a Forward Primer
  source 1..21: mol_type = other DNA
tcacacgtaggacgcatgaag

SEQ ID NO: 10 - DNA, 21 bp, synthetic construct
  misc_feature 1..21: note = Description of sequence: Primer, name: CD107a Reverse Primer
  source 1..21: mol_type = other DNA
gaagcgctccagacactcatc

(19) United States
(12) Patent Application Publication
(10) Pub. No.: US 2026/0055371 A1
(43) Pub. Date: Feb. 26, 2026

(54) DERIVING NATURAL KILLER CELLS FROM HUMAN INDUCED PLURIPOTENT STEM CELLS WITHOUT FEEDER CELLS

(71) Applicant: The Florida State University Research Foundation, Inc., Tallahassee, FL (US)

(72) Inventors: Qing-Xiang Sang, Tallahassee, FL (US); Sonia Kiran, Tallahassee, FL (US); Yu Xue, Tallahassee, FL (US); Yan Li, Tallahassee, FL (US); Drishty Badhon Sarker, Tallahassee, FL (US)

(21) Appl. No.: 19/306,489

(22) Filed: Aug. 21, 2025

Related U.S. Application Data
(60) Provisional application No. 63/686,424, filed on Aug. 23, 2024.

(51) Int. Cl. C12N 5/0783; A61K 40/15; A61K 40/42; A61P 35/00

(52) U.S. Cl. CPC C12N 5/0646; A61K 40/15; A61K 40/4224; A61P 35/00; C12N 2501/2315; C12N 2506/45

(57) ABSTRACT

The present disclosure relates to method of differentiating and/or maturing natural killer (NK) cells in the absence of feeder cells, wherein said NK cells are differentiated to various stages of maturation.

### RELATED APPLICATION

This U.S. utility application claims priority to, and the benefit of, U.S. Provisional Patent Application No. 63/686,424, filed Aug. 23, 2024, entitled “DERIVING NATURAL KILLER CELLS FROM HUMAN INDUCED PLURIPOTENT STEM CELLS WITHOUT FEEDER CELLS,” which is incorporated by reference herein in its entirety.

### STATEMENT REGARDING FEDERALLY SPONSORED RESEARCH

This invention was made with government support under CBET-1652992 and CBET-1917618 awarded by the National Science Foundation. The government has certain rights in the invention.

### REFERENCE TO SEQUENCE LISTING

The sequence listing submitted on Aug. 21, 2025, as an .XML file entitled “10850-113US1_ST26” created on Aug. 21, 2025, and having a file size of 13,741 bytes is hereby incorporated by reference pursuant to 37 C.F.R. § 1.52(e)(5).

### FIELD

The present disclosure relates to method of differentiating and/or maturing natural killer (NK) cells in the absence of feeder cells.

### BACKGROUND

Cell-based anticancer immunotherapies, such as T cell- and NK cell-based therapies, are of increasing interest for their promising therapeutic benefits. Although T cells are highly effective in killing their targets, the requirements of T cell priming and human leukocyte antigen (HLA) restriction limit their usefulness. The function of innate immune cells, such as NK cells, is not impacted by HLA restriction. They have shown promising results because of their lower risk of graft versus host disease (GVHD) and cytokine release syndrome (CRS) and higher graft versus tumor potential. However, the main concern regarding allogenic and autologous NK cell immunotherapy is the limited number of available cells, resulting in poor access to adoptive cell therapy. This limitation can be addressed by human pluripotent stem cell-derived NK cells, which can provide a robust source of off-the-shelf NK cells and can be more accessible for cancer treatment. The compositions and methods disclosed herein addresses these limitations and more.

### SUMMARY

The present disclosure provides methods of treating a cancer by administering an iPSC-derived NK cell to a subject with said cancer. The present disclosure also provides methods of generating an iPSC-derived NK cell in a feeder cell-free cell culture.

In some aspects, disclosed herein is a method of treating a cancer in a subject in need thereof, the method comprising administering to the subject a cell-based immunotherapy comprising an induced pluripotent stem cell-derived natural killer cell (iPSC-derived NK cell), wherein the iPSC-derived NK cell is prepared by (i) obtaining an induced pluripotent stem cell (iPSC); (ii) culturing the iPSC to differentiate into a hematopoietic progenitor cell; (iii) culturing the hematopoietic progenitor cell to further differentiate into a lymphoid progenitor cell; (iv) culturing the lymphoid progenitor cell to further differentiate into an immature NK cell; and (v) culturing the immature NK cell to further differentiate into the iPSC-derived NK cell, wherein the iPSC-derived NK cell is activated in a feeder cell-free cell culture, and wherein the iPSC-derived NK cell comprises comparable cytotoxicity relative to a positive control NK cell.

In some aspects, disclosed herein is a method of differentiating a human induced pluripotent stem cell (iPSC) into a mature natural killer (NK) cell in a feeder cell-free cell culture, the method comprising (i) obtaining a human induced pluripotent stem cell (iPSC); (ii) culturing the human iPSCs to differentiate into a hematopoietic progenitor cell; (iii) culturing the hematopoietic progenitor cell to further differentiate into a lymphoid progenitor cell; (iv) culturing the lymphoid progenitor cell to further differentiate into an immature NK cell; and (v) culturing the immature NK cell to further differentiate into a mature NK cell.

In some embodiments, the hematopoietic progenitor cell is a CD34⁺, CD43⁺ cell. In some embodiments, the lymphoid progenitor cell is a CD7⁺, ZBTB16⁺ cell. In some embodiments, the immature NK cell is a CD56⁺, CD16⁻ NK cell. In some embodiments, the iPSC-derived NK cell is a CD56⁺, CD16⁺ NK cell.

In some embodiments, the iPSC-derived NK cell is further activated in the presence of interleukin-15 (IL-15). In some embodiments, the iPSC-derived NK cell comprises increased expression of NKG2D, CD107a, NKp46, CD16a, CD56, or a combination thereof relative to control. In some embodiments, the iPSC-derived NK cells expressed SH2D1A, KIR2DL1, CD94, perforin, granzyme B, or a combination thereof. In some embodiments, the iPSC-derived NK comprises an increased release of interferon-gamma (IFN-γ) and a decreased release of IL-6 and tumor necrosis factor-alpha (TNF-α), or a combination thereof relative to a control.

In some embodiments, the cancer includes but is not limited to atypical teratoid rhabdoid tumor (ATRT), diffuse intrinsic pontine glioma (DIPG), lung cancer, non-Hodgkin's lymphoma, renal cell cancer, multiple myeloma, or Hodgkin's lymphoma. In some embodiments, the iPSC-derived NK cell causes degranulation in a cancer cell.

### BRIEF DESCRIPTION OF FIGURES

The accompanying figures, which are incorporated in and constitute a part of this specification, illustrate several aspects described below.

FIG. 1 shows the schematic representation of human iPSC differentiation into Natural Killer cells in five stages. Hematopoietic progenitor differentiation is achieved using EB Medium A and B, characterized by CD34^{bright} CD43^{bright} phenotype. Subsequent progression to the lymphoid progenitor stage is marked by ZBTB16^{bright} CD7^{bright} expression, facilitated by Lymphoid Progenitor Expansion Medium (SFEMII) with expansion supplement. NK differentiation involves the use of an NK Differentiation Medium comprising SFEMII, NK Supplement, and UM729. The commitment to distinct NK cell subtypes includes CD56^{bright} CD16^{-ve}, CD56^{bright} CD16^{bright}, and further maturation into CD56^{-ve} CD16^{bright} NK cells. Abbreviations: iPSC, induced pluripotent stem cell; EB, embryonic body; SFEMII, serum-free expansion medium II; UM729, pyrimido-[4,5-b]-indole derivative.

FIGS. 2A and 2B show the differentiation of hematopoietic and lymphoid progenitors from human iPSCs. FIG. 2A shows in panel (i) the phase-contrast images captured on Day 3, Day 6, and Day 8 show the progression of myeloid progenitors, while panel (ii) shows the qualitative analysis of CD34 and CD43 expression is conducted through immunocytochemistry (ICC), and panel (iii) shows the flow cytometry provides a quantitative assessment of co-staining of CD34 and CD43. FIG. 2B shows in panel (i) the phase-contrast images tracking the development of lymphoid progenitors on Day 15, Day 20, Day 25, Day 30, and Day 35. Additionally, FIG. 2B shows in panel (ii) that immunocytochemistry detects the expression of ZBTB16 and CD25 markers on Day 35. FIG. 2B also shows in panel (iii) flow cytometry quantifies ZBTB16, CD25, and CD7 on the same day of differentiation. ZBTB-16, Zinc finger and BTB domain containing 16; CD, Cluster of differentiation; Hoe, Hoechst 33342. The scale bars in FIGS. 2A and 2B are represented by 100 μm, corresponding to the magnification of 20× for ICC images and 10× for the phase contrast images.

FIGS. 3A and 3B show the commitment of lymphoid progenitors to the CD56^{bright} CD16⁻ ve phenotype and their subsequent maturation into CD56^{bright} CD16^{bright} Natural Killer (NK) cells. FIG. 3A shows in panel (i) the phase-contrast images taken on Day 45 and Day 50 reveal the differentiation of NK cells, while panel (ii) shows the immunocytochemistry images demonstrate positive expression of the CD56 and negative for CD3E+CD3G, CD94, and perforin, and panel (iii) shows the flow cytometry quantification of CD56 and CD16 indicates the commitment of lymphoid progenitors to premature NK cells. FIG. 3B further shows the differentiation results in an intermediary maturation stage (CD56^{bright} CD16^{bright}). FIG. 3B shows in panel (i) the phase-contrast images on Day 55 and Day 60 show continued differentiation, while panel (ii) shows co-staining immunocytochemistry images and panel (iii) shows the flow cytometry quantification demonstrates positive expression of CD56 and CD16 markers, signifying the progression toward mature NK cells. Additionally, examination of NKG2D, NKp30, NKp46, TRAIL, and FasL at this stage was conducted through flow cytometry. NKG2D, NKG2-D type II integral membrane protein; NKp30, Natural killer cell p30-related protein; NKp46, Natural killer cell p46-related protein; TRAIL. The scale bar in FIGS. 3A and 3B is 100 μm, corresponding to a magnification of 20× for ICC images and 10× for phase-contrast images.

FIGS. 4A and 4B shows the activation markers of CD56^{bright} CD16^{bright} iPSC-NK cells. FIG. 4A shows the immunocytochemistry data showed positive expression of the activation markers such as SH2D1A, NKG2D, CD107a, and inhibitory markers, KIR2DL and CD94 upon coculture with panel (i) CHLA-02-ATRT or panel (ii) CHLA-05-ATRT. FIG. 4B in panel (i) shows the percentage expression of the activation markers, NKG2D and NKp46, in iPSC-NK cells before and after coculture was performed using flow cytometry. FIG. 4B in panel (ii) shows the relative gene expression of NK cell markers, including NKG2D, CD107a, NKp46, CD16a and CD56 after IL-15 treatment was also measured. KIR2DL1, killer cell immunoglobulin-like receptor 2DL1; SH2D1A, SH2 domain-containing protein 1A; NKG2D, NKG2-D type II integral membrane protein. The scale bar in FIG. 4A represents 100 μm, which corresponds to a 20× magnification. The statistically significant data are represented with “*”, “**”, and “***” for p-values <0.05, <0.01, and <0.001, respectively.

FIGS. 5A and 5B shows the degranulation marker expression and cytokine production in CD56^{bright} CD16^{bright} iPSC-NK cells after exposure to ATRT cells. FIG. 5A in panel (i) shows the phase-contrast images showing coculture of iPSC-NK cells with CHLA-02-ATRT and CHLA-05-ATRT. FIG. 5A in panel (ii) shows the immunocytochemistry of degranulation marker CD107a in iPSC-NK cells after coculture with ATRT cells. FIG. 5A in panel (iii) shows the relative degranulation measurement in iPSC-NK cells before and after coculture with ATRT. “**”, p-value<0.01. FIG. 5B shows the cytokines production by human iPSC-NK cells. FIG. 5B, panel (i) Immunofluorescent images show positive TNF-alpha and IL-6 expression. FIG. 5B in panel (ii) shows the iPSC-NK cells expressing IFN-gamma after exposure to CHLA-05-ATRT are quantified via flow cytometry. FIG. 5B in panel (iii) shows the cytokines released by iPSC-NK cells after exposure to interleukins, CHLA-05-ATRT, and CHLA-02-ATRT were quantified through enzyme-linked immunosorbent assay (ELISA). “*”, p-value<0.05. ns, non-significant difference. IL-6: Interleukin 6, TNF-a: Tumor necrosis factor alpha, IFN-gamma: Interferon-gamma. iNK:CD56^{bright} CD16^{bright} iPSC-derived NK cells, ILs: IL2, IL12, IL15, IL18, and IL21. Scale bar, 100 μm. The magnification is 20× for ICC images and 10× for phase-contrast images.

FIGS. 6A and 6B show the cytotoxicity analysis of interleukins (ILs)-exposed iPSC-NK cells against ATRT cells. FIG. 6A shows the flow cytometry-based Calcein-AM assay. The quadrant plot displays the percentage expression of the live cell marker (Calcein-AM) on the x-axis and the dead cell marker (ETHD-1) on the y-axis for CHLA-02-ATRT or CHLA-05-ATRT after coculture with iPSC-NK cells. FIG. 6B shows the table depicting the percentage expression of live and dead cells as measured in FIG. 6A. The live/dead ratio is observed to be lower in the coculture of both CHLA-02-ATRT and CHLA-05-ATRT with interleukin-exposed iPSC-NK cells compared to unexposed iPSC-NK cells. ILs: IL2, IL12, IL15, IL18, and IL21.

FIGS. 7A and 7B show the decrease in the viability of cancer cells upon exposure to CD56^{bright} CD16^{bright} iPSC-NK cells. FIG. 7A shows the ratio-dependent decrease in CHLA-02-ATRT and CHLA-05-ATRT cell viability upon coculture with CD56^{bright} CD16^{bright} iPSC-NK cells; FIG. 7B shows the ratio-dependent decrease in the viability of other types of tumor cells upon coculture with CD56^{bright} CD16^{bright} iPSC-NK cells, including SF8628, a diffuse intrinsic pontine glioma cell line, G401, a kidney rhabdoid tumor cell line, and A549, a lung carcinoma cell line. INK, CD56^{bright} CD16^{bright} iPSC-NK. Statistical significance is denoted by *, **, and *** for p-values <0.05, <0.01, and <0.001, respectively.

FIGS. 8A, 8B, and 8C show the maturation of iPSC-NK cells into CD56^{-ve} CD16^{bright} phenotype. FIG. 8A in panel (i) shows the phase-contrast imaging on Day 65 and Day 70. FIG. 8A in panel (ii) shows the immunocytochemistry (ICC) images of CD56 and CD16. FIG. 8A in panel (iii) shows the flow cytometry quantification shows the highly positive expression of CD16 and low expression for the CD56 marker. FIG. 8B shows the immunocytochemistry detects the expression of CD56 and CD16 in iPSC-NK cells after activation using CHLA-02-ATRT, IL-15, and a combination of interleukins (IL2, IL12, IL15, IL18, and IL21). FIG. 8C shows the percentage expression of CD16 and CD56 under each condition remains relatively stable. Scale bar: 100 μm.

FIGS. 9A, 9B, and 9C show the characterization of the activation markers in CD56-ve CD16^{bright} iPSC-NK cells and their cytotoxicity toward ATRT cells. FIGS. 9A and 9B show the immunocytochemistry and flow cytometry data indicate the absence of expression for NKG2D and NKp46 markers. At this stage, NKG2D in iPSC-NK cells was not upregulated after coculture with CHLA-02-ATRT. FIG. 9C shows the effects of ILs-exposed iPSC-NK cells versus ILs-unexposed iPSC-NK cells on cell viability of CHLA-02-ATRT and CHLA-05-ATRT cells have no statistically significant decrease in cell viability compared to the untreated group, E:T=0:1. ns, non-significant difference. The data were verified by two independent experiments, and each experiment has triplicates (n=3).

FIGS. 10A and 10B show a repetition of flow cytometry for the reproducibility of the iPSC-NK cell differentiation protocol. It showed relatively consistent results between batches 1 (i) and 2 (ii). FIG. 10A shows the hematopoietic progenitor markers, CD34 and CD43. FIG. 10B show the immature stage, CD56^{bright} CD16^{-ve} iPSC-NK cells.

FIGS. 11A, 11B, and 11C show the differentiation stage of CD56^{bright} CD16^{bright} iPSC-NK cells. FIG. 11A shows the gating events of CD56^{bright} CD16^{bright} iPSC-NK cells in FIG. 3B (ii). FIGS. 11B and 11C show the percent expression of NKp46 (99.6%) and NKG2D (41%) in CD56^{bright} CD16^{bright} iPSC-NK cells after exposure to CHLA-05-ATRT cells were higher than NKp46 (91%) and NKG2D (30%) after exposure to CHLA-02-ATRT cells. However, NKp30 was expressed in the majority of iPSC-NK cells after exposure to either CHLA-02-ATRT or CHLA-05-ATRT. NKp46, Natural killer cell p46-related protein; NKG2D, NKG2-D type II integral membrane protein; NKp30, Natural killer cell p30-related protein.

FIGS. 12A and 12B show that assessing the efficacy of CD56^{bright} CD16^{bright} iPSC-NK cells in inducing cell death of ATRT cells. The (4,5-Dimethylthiazol-2-yl)-2,5-diphenyltetrazolium bromide (MTT) assay was employed to quantify the relative cell death of ATRT cells mediated by iPSC-NK cells across various effector-to-target ratios (E:T). The percentage of cell death was normalized to the E:T condition of 0:1.

FIGS. 13A and 13B show the Calcein-AM-based cytotoxicity assay to determine the specific cell death percentage of ATRT cells. FIG. 13A shows the CD56^{bright} CD16^{bright} iPSC-NK cells as effectors. FIG. 13B shows the NK92mi cells as effectors. Statistical significance is denoted by * and ** for p-values <0.05 and <0.01, respectively. The symbol “Δ” indicates a significant difference between ILs-exposed and unexposed groups at the same E:T ratio. ILs: IL2, IL12, IL15, IL18, and IL21.

FIGS. 14A and 14B show the examination of subpopulations existing at the CD56^{bright} CD16^{bright} stage through immunocytochemistry. FIG. 14A shows the lymphoid progenitor markers, CD7, ZBTB-16, CD5, and GATA3. Natural killer T cell or other lymphoid lineage markers, CD56 and CD3D. FIG. 14B shows the natural killer cell marker, CD56, CD16, TRAIL, NKp30, NKG2D, and CD158a. ZBTB-16, Zinc finger and BTB domain containing 16; GATA3, Trans-acting T-cell-specific transcription factor GATA3; CD3D, T-cell surface glycoprotein CD3 delta chain; TRAIL, TNF-related apoptosis-inducing ligand; NKp30, Natural killer cell p30-related protein; NKG2D, NKG2-D type II integral membrane protein. Scale bar, 100 μm.

FIG. 15 shows the gating events of CD56-Ne CD16^{bright} cells and an activation marker in CD56^{bright} CD16^{bright} cells. The gating events for FIG. 8, further maturation of iPSC-NK cells into CD56^{-ve} CD16^{bright} phenotype. Markers were detected after exposure to IL-15, ILs (IL-2, IL-12, IL-15, IL-18, and IL-21), or CHLA-02-ATRT.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “therapeutically effective amount” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, 100% or more increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, 100%, or more decrease so long as the decrease is statistically significant.

“Culture” or “cell culture” is the process by which cells are grown under controlled conditions, generally outside their natural environment. After the cells of interest have been isolated from living tissue, they can subsequently be maintained under carefully controlled conditions. These conditions vary for each cell type, but generally consist of a suitable vessel with a substrate the essential nutrients (amino or medium that supplies acids, carbohydrates, vitamins, minerals), growth factors, hormones, and gases (CO₂, O₂), and regulates the physio-chemical environment (pH buffer, osmotic pressure, temperature). Most cells require a surface or an artificial substrate to form an adherent culture as a monolayer (one single-cell thick), whereas others can be grown free floating in a medium as a suspension culture. “Cell culture” also refers to the culturing of cells derived from multicellular eukaryotes, especially animal cells, in contrast with other types of culture that also grow cells, such as plant tissue culture, fungal culture, and microbiological culture (of microbes).

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The terms “immunotherapy” and “immunotherapeutic” refers to the treatment of disease by activating or suppressing the immune system. In cancer treatment, the most effective immunotherapies are cell-based immunotherapies that utilize lymphocytes, macrophages, dendritic cells, natural killer cells, cytotoxic T lymphocytes, etc. to defend the body against cancer by targeting abnormal antigens expressed on the surface of tumor cells.

The terms “anticancer” and “anticarcinogen” refers to a substance, composition, or formula that counteracts the effects or inhibits the development of a cancerous cells and tissues.

The term “cancer” is used to address any neoplastic disease, and is not limited to epithelial neoplasms (surface and glandular cancers; such a squamous cancers or adenomas)). It is used here to describe both solid tumors and hematologic malignancies, including but not limited to epithelial (surface and glandular) cancers, soft tissue and bone sarcomas, angiomas, mesothelioma, melanoma, lymphomas, leukemias and myeloma.

The terms “cell,” “cell line” and “cell culture” include progeny. It is also understood that all progenies may not be precisely identical in DNA content, due to deliberate or inadvertent mutations. Variant progeny that have the same function or biological property, as screened for in the originally transformed cell, are included. The “host cells” used in the present invention generally are prokaryotic or eukaryotic hosts.

The term “cytotoxic” as used herein refers to the ability to kill a target cell. A cytotoxic NK cell may kill a target cell via target cell apoptosis using one or more different mechanisms including release of one or more cytotoxins or expression of a Fas ligand. In some embodiments, a cytotoxic NK cell kills a tumor cell via the release of one or more cytotoxins. “Cytotoxin” includes, but is not limited to, a perforin, a granzyme and a granulysin. Currently known granzymes includes, but are not limited to Granzyme A (GZMA), Granzyme B (GZMB), Granzyme H (GZMH), Granzyme K (GZMK), and Granzyme M (GZMM).

As used herein, “cytotoxicity” refers to the quality of being toxic to cells. Treating cells with a cytotoxic compound can result in a variety of cell fates, including necrosis (in which the cell membrane becomes compromised leading to cell lysis), senescence (in which the cell stops actively growing and dividing), or apoptosis (in which the cell activates a genetic program of controlled cell death).

The terms “treat,” “treating,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the disclosure may be applied preventively, prophylactically, palliatively or remedially. Treatments are administered to a subject prior to onset (e.g., before obvious signs of cancer), during early onset (e.g., upon initial signs and symptoms of cancer), or after an established development of cancer.

The term “differentiation” is the process in which a stem cell alters from one type to a differentiated one, in which the stem cell changes to a more specialized type. This process happens multiple times during the development of multicellular organism as it changes from a simple zygote to a complex system of tissues and cell types.

“Phenotype” refers to the set of observable characteristics or traits of an organism. This term covers an organism's morphology, or physical form and structure, its developmental processes, its biochemical and physiological properties, its behavior, and the products of behavior.

### Cell-Based Immunotherapeutics

In some aspects, disclosed herein is a cell-based immunotherapeutic comprising activated natural killer (NK) cells that are not contacted with feeder cells. In some embodiments, the cell-based immunotherapeutic is also an adoptive cell transfer therapy, wherein said therapeutics and/or therapies are cancer treatments that use a subject's immune system to fight cancer cells. Said treatments involve modifying and redirecting immune cells, either from the patient or from a donor, to target and destroy cancer cells. This approach harnesses the power of immune cells including, but not limited to NK cells, T cells, and/or macrophages to attack tumors.

Natural killer (NK) cells are innate immune cells that recognize and target non-self-cells, such as cancer and virus-infected cells, without prior sensitization. They use their activation and inhibition receptors to sense the non-self-cells and employ cell death pathways, such as the perforin-granzyme B complex and FasL-TRAIL activation pathway, to attack them. Human NK cells have multiple subtypes based on maturity and cytolytic activity—an understanding of what is particularly important for deriving functionally mature NK cells from stem cell sources. The CD56^{bright} CD16^{-ve} subset of NK cells is found mainly in secondary lymphoid tissue and comprises cytokine-producing immature NK cells. CD56^{-ve} CD16^{bright} and CD56^{bright} CD16^{bright} are cytolytic NK cells and populate human peripheral blood. The CD56^{-ve} CD16^{bright} phenotype belongs to a minor subtype of NK cells that displayed impaired cytolytic function with aging. Experimental evidence shows that CD56^{bright} NK cells give rise to CD56 ve cells during maturation.

Despite the advantages of T-cell based and NK-cell based anti-cancer immunotherapies, there remains limitations to these cell-based immunotherapies, including, but not limited to the limited number of available cells, resulting in poor access to adoptive cell therapy. One approach to overcoming these limitations is deriving NK cells from human pluripotent stem cells. Some common approaches to derive NK cells by differentiating human pluripotent stem cells use murine stromal cells as feeders. However, the derived NK cells face hurdles in their clinical application because of the presence of murine cells and associated contaminants. Therefore, the implementation of a feeder-free method to differentiate human induced pluripotent stem cells (iPSCs) into NK cells would be highly desirable. Reports showed that iPSC-derived NK cells have high proliferative potential and desired homogeneity for therapeutic use. Furthermore, since iPSCs are amenable to genome editing, modified NK cells with high target specificity, persistence, and immune activation potential are achievable. Three clinical trials are ongoing to evaluate the safety and efficacy of iPSC-NK cells in adult patients with various solid and hematologic malignancies (NCT03841110, NCT04023071, and NCT04630769). Despite ongoing therapeutic and clinical development, the use of NK cells in the treatment of pediatric cancers, especially rare pediatric brain tumors, is largely underexplored. Thus, the present disclosure provides mature, activated NK cells derived from differentiating iPSCs without requiring an interaction or contact with feeder cells.

In some embodiments, the activated NK cells are mature NK cells. In some embodiments, the activated NK cells are derived from human induced pluripotent stem cells (iPSCs). In some embodiments, the activated NK cells are CD56⁺, CD16⁺ NK cells.

In some embodiments, the human iPSCs undergo several rounds of differentiation to become a mature NK cell, such as for example a CD56⁺, CD16⁺ NK cell. In some embodiments, the CD56⁺, CD16⁺ NK cells undergo several rounds of differentiation prior to becoming CD56⁺, CD16⁺. In some embodiments, the several rounds comprise (i) obtaining an induced pluripotent stem cell (iPSC); (ii) culturing the iPSC to differentiate into a hematopoietic progenitor cell; (iii) culturing the hematopoietic progenitor cell to further differentiate into a lymphoid progenitor cell; (iv) culturing the lymphoid progenitor cell to further differentiate into an immature NK cell; and (v) culturing the immature NK cell to further differentiate into the iPSC-derived NK cell. In some embodiments, the several rounds of differentiation comprise a feeder cell-free cell culture. In some embodiments, the hematopoietic progenitor cell is a CD34⁺, CD43⁺ cell. In some embodiments, the lymphoid progenitor cell is a CD7⁺, ZBTB16⁺ cell. In some embodiments, the immature NK cell is a CD56⁺, CD16⁻ NK cell. In some embodiments, the iPSC-derived NK cell is a CD56⁺, CD16⁺ NK cell.

As used herein, a “stem cell” refers to a special type of cell comprising two important characteristics: (1) they are cells that are self-renewing, or can make more of themselves; and (2) they can become or transform into other cell types that have different functions. In other terms, a “stem cell” also refers to an undifferentiated cell of a multicellular organism, wherein the undifferentiated cell is capable of giving rise to indefinitely more cell of the same type, or upon differentiation can give rise to other cell types with different functions.

As used herein, a “pluripotent stem cell” or “PSC” refers to a type of stem cell that can differentiate into nearly any cell type including any tissue type that is derived from three basic body layers: ectoderm, endoderm, and mesoderm. Most pluripotent stem cells are embryonic stem cells or induced pluripotent stem cells. Specifically, an “induced pluripotent stem cell” or “iPSC” refers to a type of stem cell that is generated from adult somatic cells, such as for example skin cells or blood cells, by genetically reprogramming the somatic cells back to an embryonic-like state. Once in an embryonic-like state, iPSCs can differentiate into any cell type, including any cell type necessary for research or disease therapeutic purposes.

As used herein a “progenitor cell” refers to a type of cell with limited differentiation potential, meaning that said cells can develop into a limited range of cell types, unlike stem cells or pluripotent stem (i.e., induced pluripotent stem cells) which have a broader range of differentiation potential. “Progenitor cells” are an early stage of cell development, although come later in development relative to stem cells, and are also self-renewing to create more progenitor cells allowing for continued tissue maintenance and repair. Non-limiting examples of progenitor cells include hematopoietic progenitor cells, lymphoid progenitor cells, neural progenitor cells, myeloid progenitor cells, and vascular progenitor cells.

In some embodiments, the cell-based immunotherapeutic is a therapeutic composition comprising the iPSC-derived NK cell and a pharmaceutically acceptable carrier. As used herein, “pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

### Methods of Treating Cancer

The present disclosure provides methods of treating a cancer by administering an iPSC-derived NK cell to a subject with said cancer.

In some aspects, disclosed herein is a method of treating a cancer in a subject in need thereof, the method comprising administering to the subject a cell-based immunotherapy comprising an induced pluripotent stem cell-derived natural killer cell (iPSC-derived NK cell), wherein the iPSC-derived NK cell is prepared by (i) obtaining an induced pluripotent stem cell (iPSC); (ii) culturing the iPSC to differentiate into a hematopoietic progenitor cell; (iii) culturing the hematopoietic progenitor cell to further differentiate into a lymphoid progenitor cell; (iv) culturing the lymphoid progenitor cell to further differentiate into an immature NK cell; and (v) culturing the immature NK cell to further differentiate into the iPSC-derived NK cell, wherein the iPSC-derived NK cell is activated in a feeder cell-free cell culture, and wherein the iPSC-derived NK cell comprises comparable cytotoxicity relative to a positive control NK cell.

In some embodiments, the method of any preceding aspect treats, prevents, decreases, ameliorates, and/or inhibits a cancer including, but not limited to atypical teratoid rhabdoid tumor (ATRT), acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In some embodiments, the method of any preceding aspect treats, prevents, decreases, ameliorates, and/or inhibits a pediatric cancer including, but not limited to ATRT, acute lymphoblastic leukemia (ALL), Acute Myeloid Leukemia (AML), Chronic Myelogenous Leukemia (CML), Juvenile Myelomonocytic Leukemia (JMML), Hodgkin Lymphoma, Non-Hodgkin Lymphoma (NHL), astrocytoma, medulloblastoma, multiple myeloma (MM), renal cell cancer, ependymoma, diffuse intrinsic pontine glioma (DIPG), lung cancer, neuroblastoma, Wilms tumor, rhabdomyosarcoma, osteosarcoma, Ewing sarcoma, retinoblastoma, germ cell tumors (including, but not limited to tumors of the brain, abdomen, and chest), hepatoblastoma, thyroid cancer, and adrenocortical carcinoma.

ATRT is an aggressive human pediatric tumor of the central nervous system (CNS). Also known as malignant rhabdoid tumors of the brain, ATRT accounts for 20% of all CNS tumors in children under age 3. This pediatric brain tumor occurs due to the biallelic inactivating mutations of the SMARCB1 (SWI/SNF related, Matrix Associated, Actin Dependent Regulator of Chromatin, subfamily B, member 1) gene in chromosome 22. The current ATRT treatment approach includes surgery followed by chemotherapy and radiotherapy with stem cell rescue. Although almost 50% of patients receiving multimodal therapy survive at least four years, adjuvant chemotherapy and radiotherapy can lower long-term neurocognitive performance and cause reproductive and systemic dysfunction in young ATRT patients. Therefore, there is currently an urgent need to develop biologically targeted therapies that can replace existing treatment regimens to improve the long-term survival of these pediatric patients.

Toward that goal, a variety of immunotherapies are in development to treat ATRT. These immunotherapies include dendritic cell-based vaccines and T cell-based immunotherapies, which are undergoing investigation in pre-clinical and clinical trials. However, the complexity of production and inter-individual variation are some intrinsic limitations of these modalities. iPSC-derived NK cell therapy overcomes these constraints for safer clinical use. Moreover, innate NK cells have been shown to infiltrate the immune microenvironment of the majority of ATRTs, placing greater importance on their antitumor functionality against brain malignancies. Therefore, the present disclosure obtains different maturational subtypes of iPSC-NK cells and test their in vitro efficacy against malignant cells.

Thus, it has been contemplated that NK cells derived from human iPSCs through feeder-free differentiation are cytotoxic toward malignant brain rhabdoid tumor cells, and the effects can be increased via prior activation with interleukins. Herein, iPSC-NK cells were allowed to mature and develop a cytotoxic phenotype. Mature iPSC-NK cells were then cocultured with ATRT cells at various effector-to-target ratios to test their functional cytotoxicity. This iPSC-NK cell differentiation strategy to obtain functionally mature cytotoxic cells, along with their antitumor activity against ATRT cells in vitro, offers valuable insights into immunotherapeutic strategies for brain tumors.

In some embodiments, the method of any preceding aspect treats, prevents, decreases, ameliorates, and/or inhibits a pediatric cancer in a subject that is at least one day old. In some embodiments, the method of any preceding aspect treats, prevents, decreases, ameliorates, and/or inhibits a pediatric cancer that is no more than 18 years old.

In some embodiments, the method of any preceding aspect comprises the hematopoietic progenitor cell being a CD34⁺, CD43⁺ cell. In some embodiments, the method of any preceding aspect comprises the lymphoid progenitor cell being a CD7⁺, ZBTB16⁺ cell. In some embodiments, the method of any preceding aspect comprises the immature NK cell being a CD56⁺, CD16⁻ NK cell. In some embodiments, the method of any preceding aspect comprises the iPSC-derived NK cell being a CD56⁺, CD16⁺ NK cell.

In some embodiments, the method of any preceding aspect comprises activating the iPSC-derived NK cells. In some embodiments, the iPSC-derived NK cells are activated in the presence of a cytokine including, but not limited to interleukin-15 (IL-15), IL-12, and interferon-gamma (IFNγ). IL-15 is an essential cytokine for activating and enhancing NK cell functions through the promotion of NK cell development, maturation, survival, proliferation, and cytotoxicity. IL-15 is also important for increasing the expression of NK cell activating receptors. Furthermore, IL-15 achieves NK activation by regulating several downstream signaling pathways including Ras-MEK-MAPK, JAK-STAT5, and PI3K-ATK-mTOR pathways.

In some embodiments, the iPSC-derived NK cell comprises increased expression of NKG2D, CD107a, NKp46, CD16a, CD56, or a combination thereof relative to control. In some embodiments, the expression of any one of NKG2D, CD107a, NKp46, CD16a, CD56 is increased by 10%, 15%, 20%, 25%, 30%, 35%, 40%, 45%, 50%, 55%, 60%, 65%, 70%, 75%, 80%, 85%, 90%, 95%, 99%, 100%, or more relative to a control. In some embodiments, the iPSC-derived NK cell expressed SH2D1A, KIR2DL1, CD94, perforin, granzyme B, or a combination thereof. In some embodiments the iPSC-derived NK cell expresses SH2D1A, KIR2DL1, CD94, perforin, granzyme B, or a combination thereof by 10%, 15%, 20%, 25%, 30%, 35%, 40%, 45%, 50%, 55%, 60%, 65%, 70%, 75%, 80%, 85%, 90%, 95%, 99%, 100%, or more relative to a control.

In some embodiments, the method of any preceding aspect leads to the iPSC-derived NK cell comprising an increased release of interferon-gamma (IFN-γ) and a decreased release of IL-6 and tumor necrosis factor-alpha (TNF-α), or a combination thereof relative to a control. In some embodiments, the iPSC-derived NK cell comprises 10%, 15%, 20%, 25%, 30%, 35%, 40%, 45%, 50%, 55%, 60%, 65%, 70%, 75%, 80%, 85%, 90%, 95%, 99%, 100%, or more increase of IFN-γ relative to a control. In some embodiments, the iPSC-derived NK cell comprises 10%, 15%, 20%, 25%, 30%, 35%, 40%, 45%, 50%, 55%, 60%, 65%, 70%, 75%, 80%, 85%, 90%, 95%, 99%, 100%, or more decrease of IL-6 relative to a control. In some embodiments, the iPSC-derived NK cell comprises 10%, 15%, 20%, 25%, 30%, 35%, 40%, 45%, 50%, 55%, 60%, 65%, 70%, 75%, 80%, 85%, 90%, 95%, 99%, 100%, or more decrease of TNF-α relative to a control.

In some embodiments, the method of any preceding aspect comprises the iPSC-derived NK cell causing degranulation within a cancer cell.

In some embodiments, the cell-based immunotherapeutic of any preceding aspect or a composition thereof may be administered in such amounts, time, and route deemed necessary in order to achieve the desired result. The exact amount of the cell-based immunotherapeutic of any preceding aspect or a composition thereof will vary from subject to subject, depending on the species, age, and general condition of the subject, the severity of the cancer, the particular cell-based immunotherapeutic of any preceding aspect or a composition thereof, its mode of administration, its mode of activity, and the like. The cell-based immunotherapeutic of any preceding aspect or a composition thereof is preferably formulated in dosage unit form for ease of administration and uniformity of dosage. It will be understood, however, that the total daily usage of the cell-based immunotherapeutic of any preceding aspect or a composition thereof will be decided by the attending physician within the scope of sound medical judgment. The specific therapeutically effective dose level for any particular subject will depend upon a variety of factors including the cancer being treated and the severity of said cancer; the activity of the cell-based immunotherapeutic of any preceding aspect or a composition thereof employed; the specific cell-based immunotherapeutic of any preceding aspect or a composition thereof employed; the age, body weight, general health, sex and diet of the patient; the time of administration, route of administration, and rate of excretion of the specific cell-based immunotherapeutic of any preceding aspect or a composition thereof employed; the duration of the treatment; drugs used in combination or coincidental with the specific cell-based immunotherapeutic of any preceding aspect or a composition thereof employed; and like factors well known in the medical arts.

The cell-based immunotherapeutic of any preceding aspect or a composition thereof may be administered by any route. In some embodiments, the cell-based immunotherapeutic of any preceding aspect or a composition thereof is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the cell-based immunotherapeutic of any preceding aspect or a composition thereof (e.g., its stability in the environment of the subject's immune system), the condition of the subject (e.g., whether the subject is able to tolerate oral administration), etc.

The exact amount of cell-based immunotherapeutic of any preceding aspect or a composition thereof required to achieve a therapeutically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In some embodiments, the method of any preceding aspect further comprises administering an additional anti-cancer agent. Non-limiting examples of an anti-cancer agent includes interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)), PD-L1 inhibitor including, but is not limited to Atezolizumab, Avelumab, Durvalumab, LY3300054 (Eli Lilly and Company), and monoclonal antibodies or monoclonal antibody conjugates that act as a PD-L1 inhibitor, PD-1 inhibitor including, but is not limited to pembrolizumab, Nivolumab, Cemiplimab and monoclonal antibodies or monoclonal antibody conjugates that act as a PD-1 inhibitors, and CTLA-4 inhibitor including, but is not limited to Ipilimumab, AGEN1884 and monoclonal antibodies or monoclonal antibody conjugates that act as a CTLA-4 inhibitor.

Exemplary chemotherapeutic agents include, but are not limited to, anti-estrogens (e.g. tamoxifen, raloxifene, and megestrol), LHRH agonists (e.g. goserelin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonucleotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERBR, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, leucovorin, pemetrexed, cyclophosphamide, dacarbazine, procarbazine, prednisolone, dexamethasone, campathecin, plicamycin, asparaginase, aminopterin, methopterin, porfiromycin, melphalan, leurosidine, leurosine, chlorambucil, trabectedin, discodermolide, caminomycin, aminopterin, and hexamethyl melamine.

In some embodiments, the method of any preceding aspect further comprises administering an additional medical compound/composition, including but not limited an antibiotic, an anti-inflammatory compound, an anesthetic, a sedative, and an analgesic. The additional antibiotic composition comprises any one type of antibiotic including, but not limited to penicillins (including, but not limited to amoxicillin, clavulanate and amoxicillin, ampicillin, dicloxacillin, oxacillin, and penicillin V potassium), tetracyclins (including, but not limited to demeclocycline, doxycycline, eravacycline, minocycline, omadacycline, sarecycline, and tetracycline), cephalosporins (cefaclor, cefadroxil, cefdinir, cephalexin, cefprozil, cefepime, cefiderocol, cefotaxime, cefotetan, ceftaroline, cefazidme, ceftriaxone, and cefuroxime), quinolones (also referred to as fluoroquinolones include, but are not limited to ciprofloxacin, delafloxacin, levofloxacin, moxifloxacin, and gemifloxacin), lincomycins (including clindamycin and lincomycin), macrolides (including, but not limited to azithromycin, clarithromycin, erythromycin, and fidaxomicin (ketolide)), sulfonamides (including sulfamethoxazole and trimethoprim, and sulfasalazine), glycopeptides (including, but not limited to dalbavancin, oritavancin, telavancin, and vancomycin), aminoglycosides (including, but not limited to gentamicin, tobramycin, and amikacin), carbapenems (including, but not limited to imipenem and cilastatin, meropenem, and ertapenem), and topical antibiotics (including, but not limited to neomycin, bacitracin, polymyxin B, and praxomine) used alone or in combination. In some embodiments, the anti-inflammatory compound is a non-steroidal anti-inflammatory compound including, but is not limited to aspirin, ibuprofen, ketoprofen, naproxen, steroids, glucocorticoids (including, but not limited to betamethasone, budesonide, dexamethasone, hydrocortisone, hydrocortisone acetate, methylprednisolone, prednisolone, prednisone, and triamcinolone), methotrexate, sulfasalazine, lefunomide, anti-Tumor Necrosis Factor (TNF) medications, cyclophosphamide, and mycophenolate. In some embodiments, the anesthetic includes, but is not limited to chloroprocaine, procaine, tetracaine, lidocaine, bupivacaine, ropivacaine, mepivacaine, and levobupivacaine. In some embodiments, the sedative includes, but is not limited to barbiturates, benzodiazepines, nonbenzodiazepines hypnotics, antihistamines, muscle relaxants, opioids, and methaqualone, or derivatives thereof.

### Methods of Making Cell-Based Immunotherapeutics

The present disclosure also provides methods of generating an iPSC-derived NK cell in a feeder cell-free cell culture.

In some aspects, disclosed herein is a method of differentiating a human induced pluripotent stem cell (iPSC) into a mature natural killer (NK) cell in a feeder cell-free cell culture, the method comprising (i) obtaining a human induced pluripotent stem cell (iPSC); (ii) culturing the human iPSCs to differentiate into a hematopoietic progenitor cell; (iii) culturing the hematopoietic progenitor cell to further differentiate into a lymphoid progenitor cell; (iv) culturing the lymphoid progenitor cell to further differentiate into an immature NK cell; and (v) culturing the immature NK cell to further differentiate into a mature NK cell.

In some embodiments, the method of any preceding aspect comprises the hematopoietic progenitor cell being a CD34⁺, CD43⁺ cell. In some embodiments, the method of any preceding aspect comprises the lymphoid progenitor cell being a CD7⁺, ZBTB16⁺ cell. In some embodiments, the method of any preceding aspect comprises the immature NK cell being a CD56⁺, CD16⁻ NK cell. In some embodiments, the method of any preceding aspect comprises the iPSC-derived NK cell being a CD56⁺, CD16⁺ NK cell.

In some embodiments, the method of generating an iPSC-derived NK cell comprises human iPSCs undergoing several rounds of differentiation to become a mature NK cell, such as for example a CD56⁺, CD16⁺ NK cell. In some embodiments, the method of generating an iPSC-derived NK cell comprises the CD56⁺, CD16⁺ NK cells undergoing several rounds of differentiation prior to becoming CD56⁺, CD16⁺.

In some embodiments, the method of any preceding aspect comprises activating the iPSC-derived NK cells. In some embodiments, the iPSC-derived NK cells are activated in the presence of a cytokine including but not limited to interleukin-15 (IL-15), IL-12, and interferon-gamma (IFNγ).

In some embodiments, the method of any preceding aspect comprises generating the iPSC-derived NK cell in a cell culture in the absence of feeder cells. In some embodiments, the method of any preceding aspect comprises generating the iPSC-derived NK cells without contact or interaction with a feeder cell.

In some embodiments, the method of any preceding aspect comprises the methods and reagents of Example 1 of the present disclosure. In some embodiments, the method of any preceding aspect comprises culturing the iPSCs and the iPSC-derived NK cells in a differentiation medium. In some embodiments, the differentiation medium comprises STEMdiff™ Hematopoietic EB Basal Medium with Supplement A and/or Supplement B. In some embodiments, the differentiation medium comprises StemSpan™ SFEM II medium and StemSpan™ NK Cell Differentiation Supplement (100×) and UM729. The UM729 is a pyrimido-[4,5-b]-indole derivative, which enhances the self-renewal of human hematopoietic stem cells during the differentiation to NK cells.

In some embodiments, the differentiation medium comprises a sodium ion concentration optimized to ensure differentiation of the iPSC into a mature, functional, and activated NK cell. In some embodiments, the differentiation medium comprises a potassium ion concentration optimized to ensure differentiation of the iPSC into a mature, functional, and activated NK cell. In some embodiments, the differentiation medium comprises a total amino acid concentration optimized to ensure differentiation of the iPSC into a mature, functional, and activated NK cell, wherein the total amino acid concentration comprises one or more amino acids selected from alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

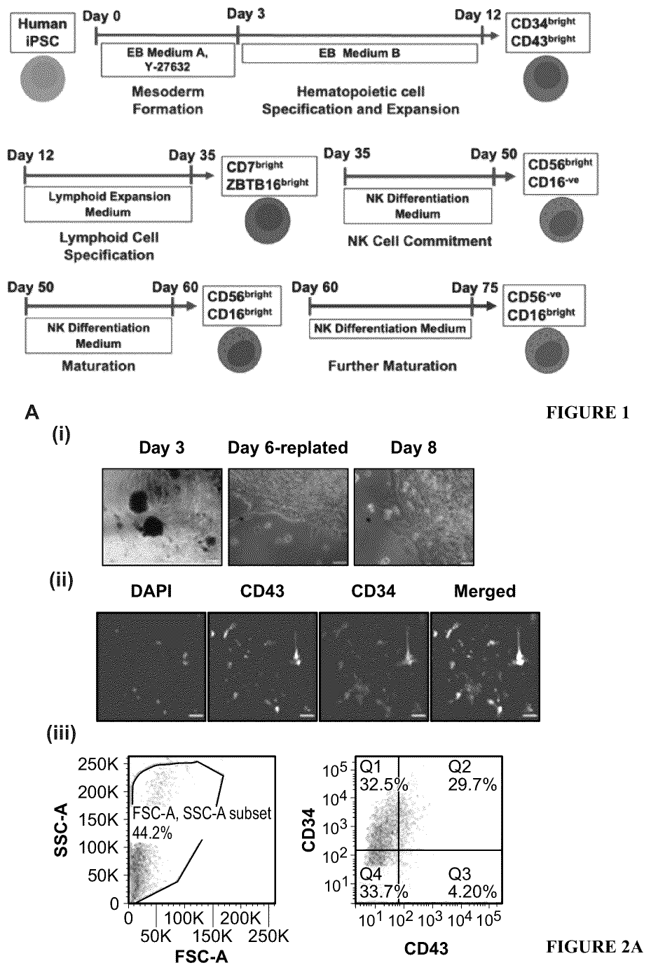

In some embodiments, the method of any preceding aspect comprises implementing the differentiation protocol of FIG. 1. In some embodiments, the method of any preceding aspect requires at least 12 days to differentiate to the human iPSC into a NK cell. In some embodiments, the method of any preceding aspect requires 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more days to differentiate the human iPSC into a NK cell.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Feeder-Free Differentiation of Human iPSCs into Natural Killer Cells

### Materials and Methods

Human iPSC and Cancer Cell Culture

The Episomal (Epi)-iPSCs (Thermo Fisher, Cat #A18945) were used for NK differentiation. These cells are derived from CD34⁺ cord blood using a three-plasmid episomal system that codes for seven reprogramming factors (SOX2, OCT4, KLF4, MYC, NANOG, LIN28, and SV40 T antigen; SOKMNLT). The cells were plated with a surface density of 1×10⁶ cells per well on a 6-well plate coated with Matrigel matrix (Life Technologies). They were maintained in mTeSR plus serum-free medium (Stem Cell Technologies, Inc). For the initial 24 h, growth maintaining factor called Rho-associated protein kinase (ROCK) inhibitor Y27632 (10 μM, Sigma) was added.

Herein, two distinct types of ATRT cell lines, namely CHLA-05-ATRT (ATCC™ CRL-3037™, ATCC) and CHLA-02-ATRT (ATCC™ CRL-3020™, ATCC), were used to test the cytotoxic effect of iPSC-NK cells. The ATRT cells were cultured in suspension on a non-treated surface using Gibco Dulbecco's Modified Eagle Medium: Nutrient Mixture F-12 (DMEM/F12, Thermo Fisher Scientific, Waltham, MA, USA) supplemented with 2% B-27, 20 ng/ml of epidermal growth factor (EGF, 78,006.1, STEMCELL Technologies, Vancouver, BC, Canada), and 20 ng/ml of fibroblast growth factor (FGF)-2 (78,003.1, STEMCELL Technologies) as mentioned previously. A549 human adenocarcinoma lung epithelial cells were purchased from ATCC (CCL-185™) and were cultured in Gibco DMEM high glucose media (Thermo Fisher Scientific, Waltham, MA, USA) with 10% fetal bovine serum (FBS). G401 kidney rhabdoid tumor cells were also purchased from ATCC (CRL-1441™) and were cultured in McCoy's 5A Medium (ATCC, 30-2007™) supplemented with 10% FBS. SF8628 Human DIPG H3.3-K27 M cell line (Sigma-Aldrich, Cat #SCC127) was grown according to the supplier's protocol. All cell lines were cultured at 37° C. and 5% CO₂ in an incubator.

Differentiation of Human iPSC's into NK Cells.

The differentiation stages of human iPSCs into NK cells were achieved using reagents from the StemSpan NK Cell generation kit (Stem cell technologies). The NK cell differentiation protocol provided by the kit manufacturer was taken as a guide to obtain three phenotypes of NK cells: CD56^{bright} CD16^{-ve}, CD56^{bright} CD16^{bright}, and CD56^{-ve} CD16^{bright} NK cells. To initiate the feeder free-differentiation, the human iPSCs were seeded in the Ultra-Low Attachment (ULA) 24-well plates (Corning Inc., Corning, NY) at 3×10⁵ cells/well in differentiation medium composed of STEMdiff™ Hematopoietic EB Basal Medium with Supplement A (5 μL/mL) in the presence of ROCK inhibitor Y27632 (10 μM). On day 3, the media was replaced with STEMdiff™ Hematopoietic EB Basal Medium with Supplement B (100 μL/mL). The culture was incubated for 3 days at 37° C. with the half media change within 2 days. On day 6, the EBs were dissociated with Accumax (Stem cell technologies) and replated at the density of 1×10⁵ cells/well on a non-tissue culture-treated 6-well plate for another 6 days. Half of the media B was replaced with fresh media every two days till day 12. At this stage, the cells were studied for the expression of markers associated with hematopoiesis such as CD34 and CD43. On day 12, the progenitor cells were directed toward the lymphocyte lineage, where the cells were harvested, dissociated with Accutase (Stem cell technologies), and replated 1×10⁵ cells/well on a tissue culture-treated 6-well plate pre-coated with Matrigel. The lymphocyte differentiation media was prepared by mixing StemSpan™ SFEM II medium with StemSpan™ Lymphoid Progenitor Expansion Supplement (10×). The lymphocyte differentiation media was added to each well of 6-well plate and incubated at 37° C. Half of the media was replaced with fresh media every 2-3 days, depending on the rate of cell proliferation. Upon confluency, the cells were detached with Accutase and replated on a T-25 flask (6×10⁵ cells/well) coated with the Matrigel and were expanded till day 30. Upon lymphoid progenitor differentiation and expansion, the cells were analyzed for the progenitor and lymphoid markers such as CD5, CD7, ZBTB16, and others. After 16 days of lymphoid expansion, the cells were further cultured for the commitment toward NK cells using differentiation medium (StemSpan™ SFEM II medium and StemSpan™ NK Cell Differentiation Supplement (100×)) and UM729. The UM729 is a pyrimido-[4,5-b]-indole derivative, which enhances the self-renewal of human hematopoietic stem cells during the differentiation to NK cells. For the NK cell commitment, the Lymphoid progenitors were replated on a non-treated 6-well plate at 1×10⁵ cells/well and cultured in the NK cell differentiation media with the supplement and UM729 (1 μM). The media was changed every 2-3 days, depending on the cell density. On day 50, the cells detached with Accutase and analyzed for the expression of NK cell-specific markers such as CD56 and CD16. The cells initially obtained CD56^{bright} CD16^{-ve} immature phenotype on day 50, but with further differentiation, they matured into CD56^{bright} CD16^{bright} phenotype. Further maturation led to CD56^{-ve} CD16^{bright} NK cells. The schematic diagram of the differentiation protocol is shown in FIG. 1.

### Calcein-AM Release Assay to Measure Cell Lysis.

The differentiated iPSC-NK cells were cocultured with CHLA-02-ATRT and CHLA-05-ATRT tumor cell lines at various effector-to-target (E:T) ratios before measuring the cytotoxicity of NK cells via Calcein-AM-based assay. The target cells were plated at the density of 10,000 cells/well in the 96-well plates and were incubated at 37° C. for 30 min with 15 μM of Calcein-AM (Life Technologies C3099). Calcein-AM-labeled cell lines were cocultured with NK cells in a surface-treated 96-well plate for 4 h at 37° C. at three different E:T ratios (1:1, 3:1, and 6:1). For the measurement of spontaneous release, all target cells were incubated without NK cells. The total release was achieved by adding 4% Triton™ X-100 (Sigma-Aldrich) to the target cells. After the incubation, the supernatant was collected and transferred to a 96-well plate to measure the Calcein-AM release at 485 nm using the spectrophotometer (SpectraMax iD5). The percentage of specific lysis was calculated according to the following formula:

Testrelease-Mediumfluorescence-Spontaneousrelease-Mediumfluorescence/Totalrelease-Trito nX100fluorescence-Spontaneousrelease-Mediumfluorescence×100.

The spontaneous release was used as the negative control, whereas Triton X-100 fluorescence was used as the positive control.

### (4,5-Dimethylthiazol-2-yl)-2,5-diphenyltetrazolium bromide (MTT) Assay

The iPSC-NK cells were cocultured in a 96-well plate with ATRT cells at various E: T ratios for 4 h. After coculture, the effector cells in suspension were removed, and attached target cells were washed with phosphate-buffered saline (PBS). MTT stock solution (5 mg/mL) was diluted in cell culture media at 1:10 and was added to 96-well plates containing tumor cells. The culture was incubated for 30 min at 37° C., and the cells were checked every 10 min till they turned purple. Once the cells turned purple, the supernatant was discarded, and the cells were washed with PBS. The formazan crystals were hydrolyzed by adding DMSO, and the absorbance of dissolved formazan was measured at 550 and 590 nm using a spectrophotometer. ATRT cells not exposed to iPSC-NK cells were used as negative controls, while cells treated with Triton X-100 were used as positive controls. The cell viability and relative percentage cell lysis data were calculated using the following formulae:

Relativepercentagecellviability(Y)=((viabilityoftargetcellsexposedtoNKcells)/(viabilityofunexpo sedtargetcells)×100);Relativepercentagecelllysis=100−Y

### Immunocytochemistry (ICC).

The cells were plated on the wells of a Matrigel-coated 96-well plate at the density of 8000 cells/well for 24 h. The cells were then fixed with 10% neutral buffered formalin (VWR) for 45 min at room temperature (RT). After the fixation, for intracellular markers, the cells were permeabilized with 0.5% Triton X-100 for 10 min. To avoid non-specific bindings, the cells were blocked with a blocking buffer (2% fetal bovine serum in PBS) for 45 min. For each marker, primary antibody staining was performed overnight at 4° C. For the secondary staining, Alexa Fluor 488 goat anti-mouse IgG and IgM or Alexa Fluor 594 goat anti-rabbit IgG (H+L) (Life Technologies) were used. Hoechst 33342 (blue) was used to stain nuclei. A fluorescent microscope (Olympus IX70, Melville, NY) was used to take immunofluorescent images. Captured images were analyzed using ImageJ software.

### Intra- and Extracellular Marker Quantification Using Flow Cytometry.

The cells were detached using Accumax and fixed using 10% neutral buffered formlin (VWR) for 45 min at RT and then washed 2-3 times with PBS. For intracellular markers, the cells were permeabilized with 100% cold methanol for 10 min. The cells were then blocked with a Blocking buffer (2% FBS in PBS) at RT for 45 min. The primary antibodies were added to all single- and double-stained cell samples and incubated overnight at 4° C. After 2-3 PBS washes, the secondary antibody Alexa Fluor 488 goat anti-mouse IgG and IgM, Alexa Fluor 568 goat anti-mouse IgG, or Alexa Fluor 594 goat anti-Rabbit IgG (H+L) (Life Technologies) were added and incubated for 1 h at RT. After 2-3 washes, the samples were run through a BD FACSCanto™ II flow cytometer (Becton Dickinson). While running the double-stained samples the compensation feature of the instrument was used to minimize the spectral overlap. The results were then analyzed against isotype controls using FlowJo software.

### Reverse Transcription-Polymerase Chain Reaction (RT-PCR).

Total mRNA was extracted from iPSC-NK cells using RNeasy™ Mini Kit (Qiagen, Valencia, CA). The isolated RNA was concentrated and cleaned using the DNA-Free RNA Kit (Zymo, Irvine, CA). About 1 μg of total RNA was used for reversed transcription. The RNA was anchored oligo-dT primers and treated with Superscript™ III (Invitrogen, Carlsbad, CA). The Primer-BLAST tool was used to design the primers (Table 2). The melting points of these primers were checked using NetPrimer. The β-actin gene was used as an endogenous control and for the normalization of expression levels of all the other markers. The RT-PCR experiment was done using an ABI7500 instrument (Applied Biosystems, Foster City, CA) and SYBR Green PCR Master Mix (QuantaBio, Beverly, MA). The amplification was done as follows: 95° C. (10 min), 40 cycles of 95° C. (15 s), 55° C. (30 s), and 68° C. (30 s). The Cₜ values of the target genes were normalized to the Cₜ values of the endogenous β-actin control. The corrected Ct values were then compared for the IL15-exposed iPSC-NK and unexposed iPSC-NK cells. Fold changes in gene expression were calculated using the comparative Cₜ method: 2^{-(ΔCt sample-ΔCt control)} to obtain relative expression levels.

### Live/Dead Quantification Via Flow Cytometry.

The Live/Dead™ staining kit (Molecular Probes) was used to measure live/dead cells. The ATRT cells were cocultured with NK cells (both interleukins-exposed and unexposed) for 7 h in the coculture media (1:1 ratio of DMEM/F-12 supplemented with 2% B-27, 20 ng/ml of epithelial growth factor (EGF) and 20 ng/ml of fibroblast growth factor (FGF)-2: RPMI media with 10% FBS). After coculture, ATRT cells were washed twice with PBS and suspended in Accumax for the formation of a single-cell suspension. The cells were incubated in DMEM/F-12 containing 5 μM Calcein-AM (green) and 4 μM ethidium homodimer I (red) in the dark for 20-25 min at RT. The samples were then run using BD FACSCanto™ II flow cytometer (Becton Dickinson) and analyzed against compensation controls using FlowJo software.

### Degranulation and Cytokine Expression.

iPSC-NK cells were cocultured with CHLA-02-ATRT and CHLA-05-ATRT target cells at the E:T ratio of 2:1 in a 24-well plate for 4 h at 37° C. The cell surface expression of the degranulation marker CD107a was measured using ICC and flow cytometry following the published protocols [49,50]. For the measurement of intracellular cytokine expression in iPSC-NK cells, the cells were also cocultured with the ATRT cells for 4 h at 37° C. at the same E:T ratio. The expression of cytokines TNF-α and IL-6 was captured using ICC. The IFN-γ expression was quantified using flow cytometry.

### ELISA for Cytokine Release Measurement.

The secretion of TNF-α, IL-6, and IFN-γ was measured through ELISA immune assays. The iPSC-NK cells were plated in the 96-well plate at the density of (1×10⁵ cells/mL) overnight. The next day, the iPSC-NK cells were exposed to ILs, CHLA-02-ATRT (E:T=2:1), CHLA-05-ATRT (E:T=2:1) for another 12 h. The unexposed condition was also cultured for the same period. The consumed media was taken out and centrifuged down at 400×g for 5 min. Then from each condition, 100 μL of supernatant was taken out for ELISA assay. The samples (Unexposed, IL-exposed, CHLA-02-ATRT exposed, and CHLA-05-ATRT exposed) and the standards were added to the pre-coated 96-well plate, and the assay was performed using the ELISA kit manufacturer's protocol. The ELISA assay for IL-6 and IFN-γ was performed using the protocol and the kit from BioLegend. For TNF-α, the ELISA protocol and kit from R&D Systems was used.

### Statistical Analysis.

Each experiment was carried out at least two times with triplicates. The representative experiments were presented, and the results were expressed as [mean±standard deviation]. To measure the statistical significance, one-way ANOVA followed by Fisher's LSD post hoc tests were performed. A p-value<0.05 was considered statistically significant.

### Results

Feeder-Free Differentiation of Human iPSCs Yielded Distinct Maturational Stages of NK Cells.

Human Epi-iPSCs were differentiated into three major NK cell subtypes that are commonly seen in human peripheral blood, i.e., CD56^{bright} CD16-ve, CD56^{bright} CD16^{bright}, and CD56-ve CD16^{bright} (FIG. 1). The feeder-free sequential differentiation process was performed in five stages. In stage I, CD34^{bright} and CD43^{bright} hematopoietic progenitors were obtained from iPSCs around day 12. At the end of stage II, cells expressed markers of innate lymphoid progenitors such as ZBTB-16 (Zinc finger and BTB domain-containing protein-16) around day 35, indicating innate lymphoid cell fate decision. Once the progenitors showed lineage specification into innate immune cells, NK differentiation media was used to attain immature phenotype (CD56^{bright} CD16^{-ve}) of NK cells around day 50, displaying their commitment to the NK cell lineage after stage III. CD56^{bright} CD16^{-ve} NK cells were further differentiated and matured into CD56^{bright} CD16^{bright} subtype (maturation stage 1) around day 60 (end of stage IV). Additional maturation (stage V) of the derived cells to day 75 gave rise to CD56^{-ve} CD16^{bright} cells (maturation stage 2) (FIG. 1).

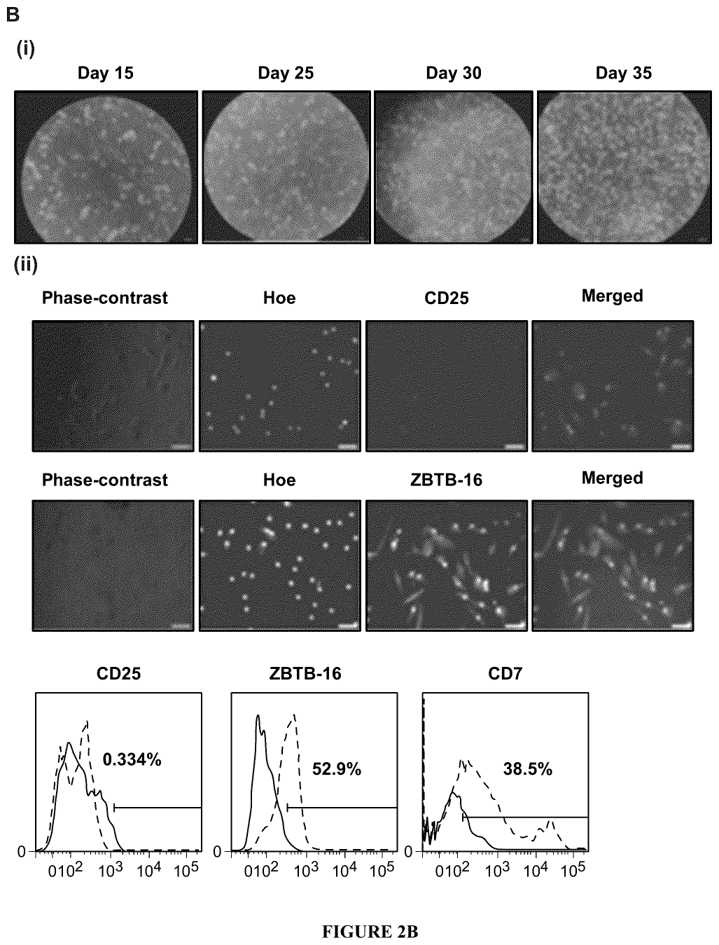

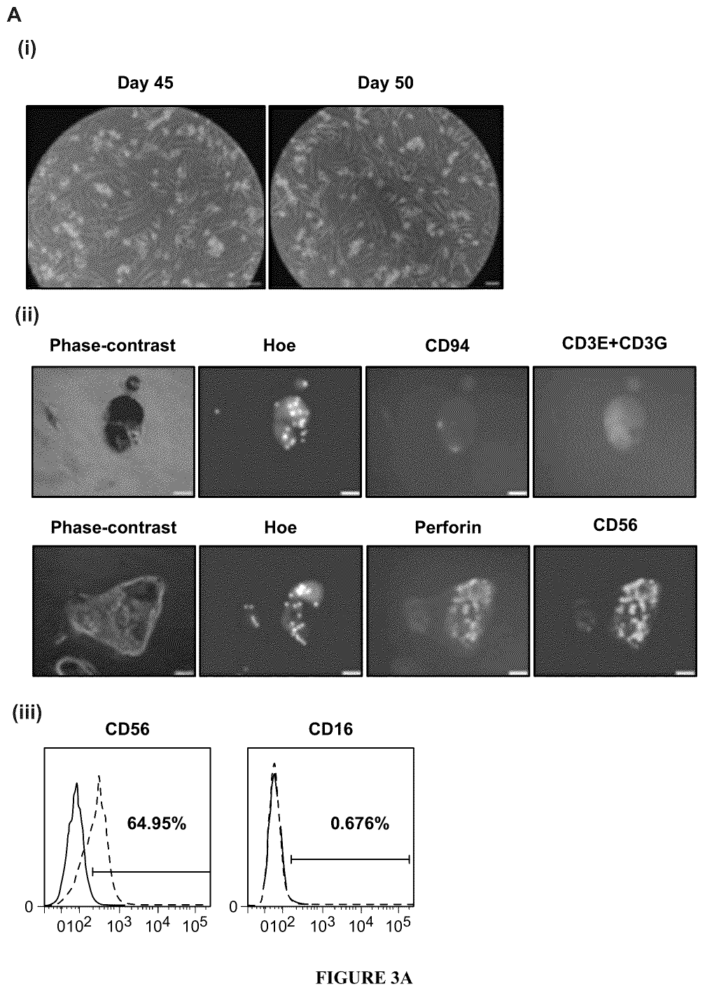

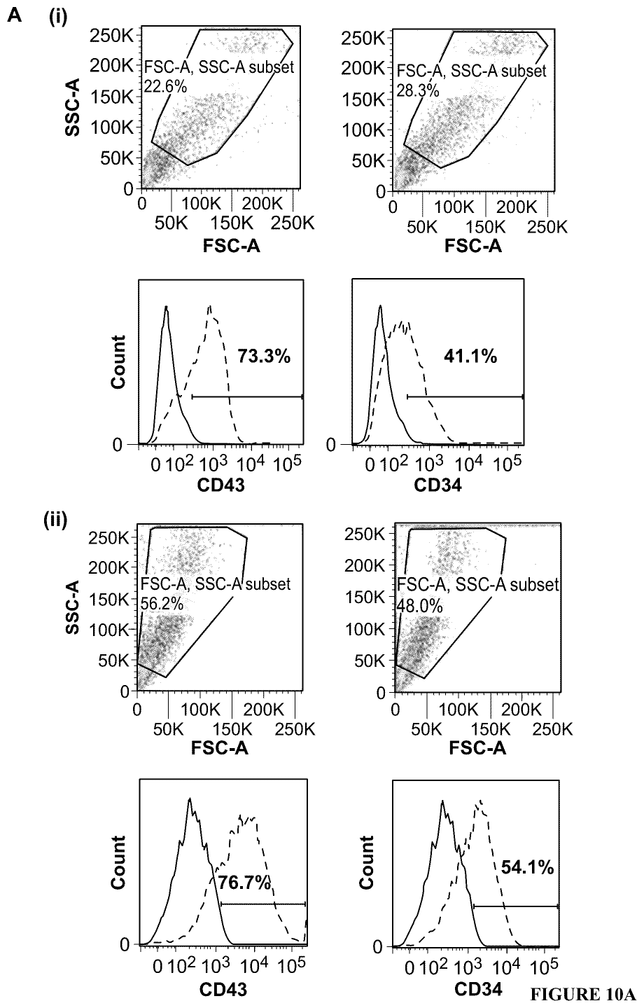

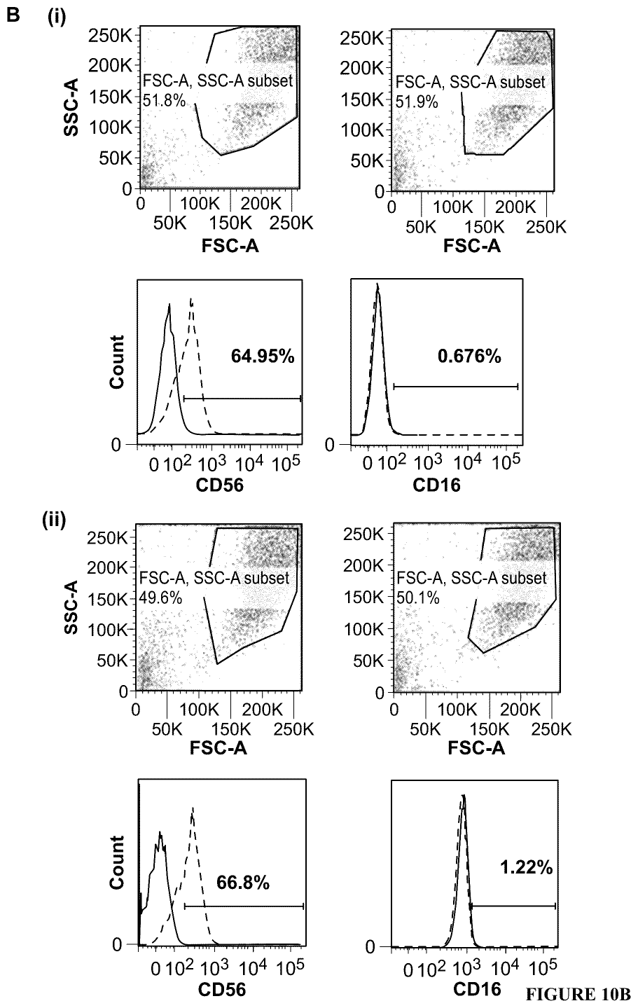

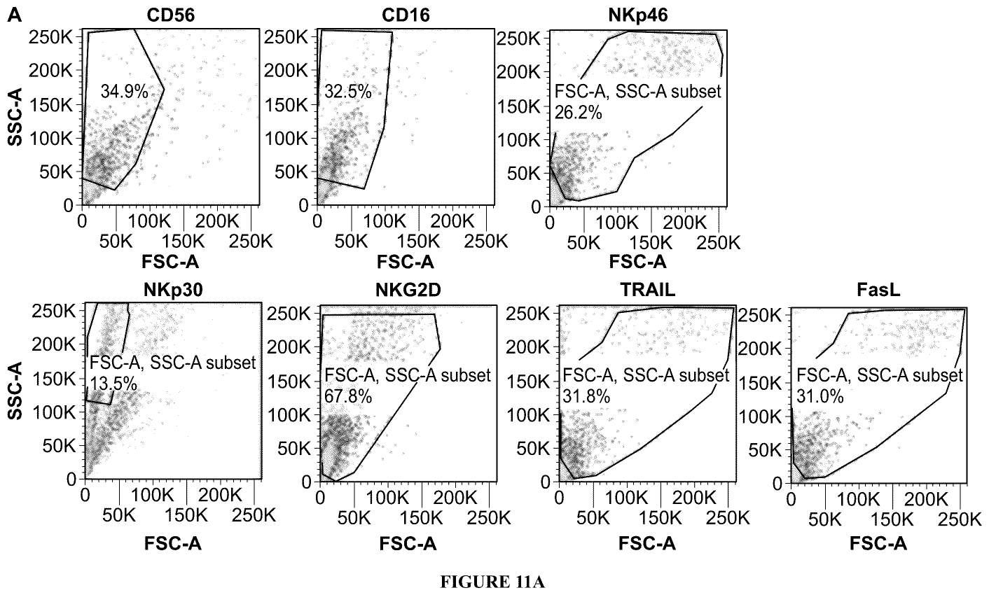

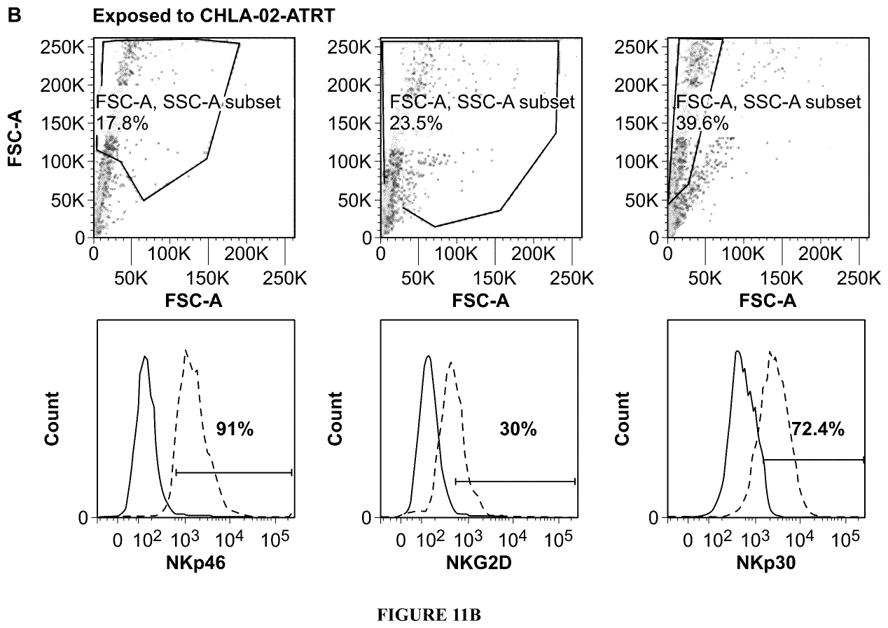

The morphological changes of the cells were monitored throughout the differentiation. The human Epi-iPSCs aggregated to form embryoid bodies (EBs) on day 3. On day 6, the EBs were replated on tissue culture-treated plates and proliferated as hematopoietic progenitor cells. The morphologies on day 3, day 6, and day 8 are shown in FIG. 2A, panel (i). CD34 and CD43 are cell surface markers of hematopoietic progenitors. The immunocytochemistry results shown in FIG. 2A, panel (ii) are positive for CD34 and CD43, indicating successful induction of iPSCs toward hematopoietic progenitors. To validate the results, 12-day progenitors were analyzed by flow cytometry. 29.7% of the cells co-expressed CD34 and CD43 (FIG. 2A, panel (iii)), and the percentage of CD34^{bright} and CD43^{bright} cells varied between 41.1-54.1% and 73.3-76.7%, respectively (FIG. 10A). The results indicate the achievement of hematopoietic progenitor phenotype by the cells that can commit to lymphoid precursors.

The next stage of differentiation was lymphoid progenitor formation. The cells were replated on Matrigel to support lymphoid lineage induction and expansion. The morphology of the cells was captured every five days starting from day 15 (FIG. 2B, panel (i)). At this stage of differentiation, cells have low expression of CD25 but high expression of ZBTB-16 and CD7. A high expression of ZBTB-16 indicates the development of innate lymphoid lineages, which was confirmed by the fluorescence imaging data shown in FIG. 2B, panel (ii). The percentage expression of CD25, ZBTB-16, and CD7 markers in lymphoid precursors on day 33 was quantified by flow cytometry as 0.33%, 52.9%, and 38.5%, respectively (FIG. 2B, panel (iii)).

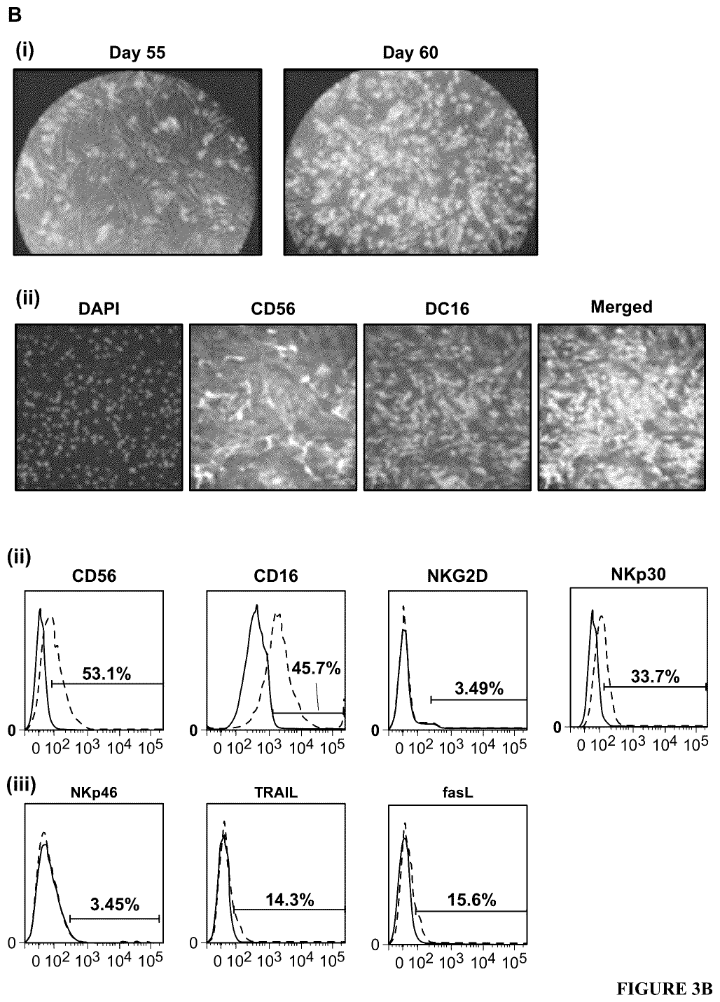

The third stage of differentiation involves the commitment of lymphoid cells to NK cells. CD56 is a well-known marker specific to NK cells, and its co-expression with CD16 signifies whether the differentiated cells are immunoregulatory or cytotoxic. By day 50, the cells had successfully committed toward NK fate (FIG. 3A, panel (i)). The immunofluorescent images showed high expression of CD56 but negative results for CD94 and CD3E+CD3G (FIG. 3A, panel (ii)); Flow cytometry results confirmed high CD56 expression (64.95-66.8%) and low CD16 expression (0.67-1.22%) (FIG. 3A, panel (iii) and FIG. 10B). These results indicated the lineage specification of lymphoid progenitors toward the CD56^{bright} CD16^{-ve} subset of NK cells. The NK cells in this subset are immature with immunomodulatory phenotype. The immature subset of NK cells has been shown to have low cytotoxicity; therefore, they do not secret perforins and are negative for CD3 and CD94 markers.

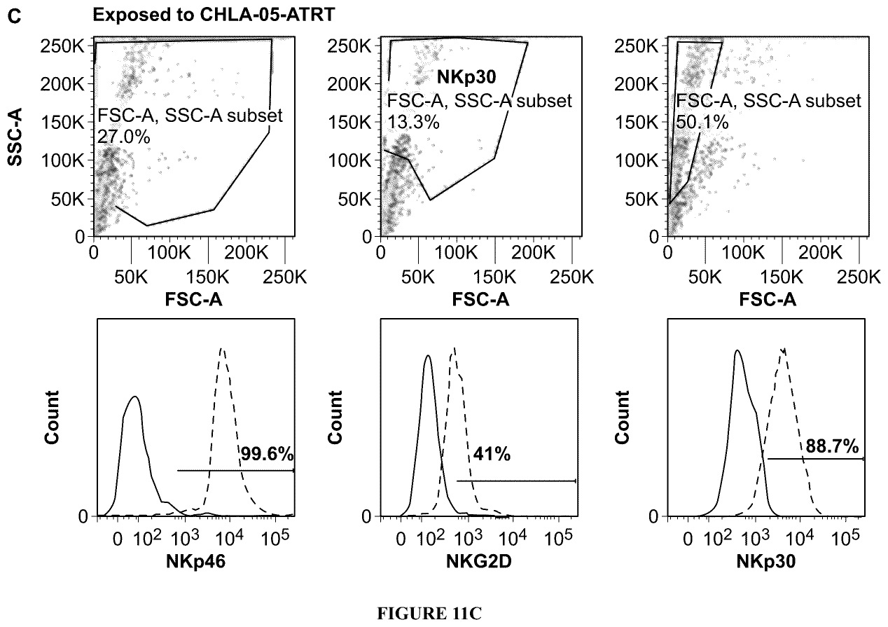

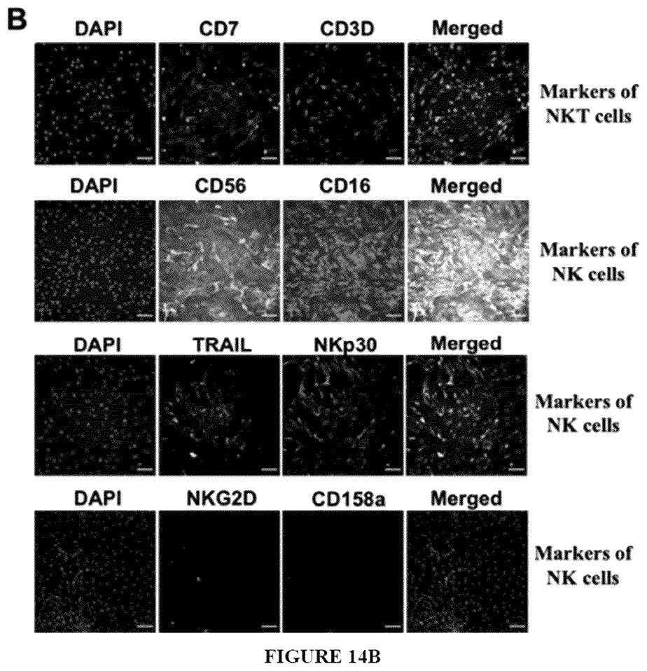

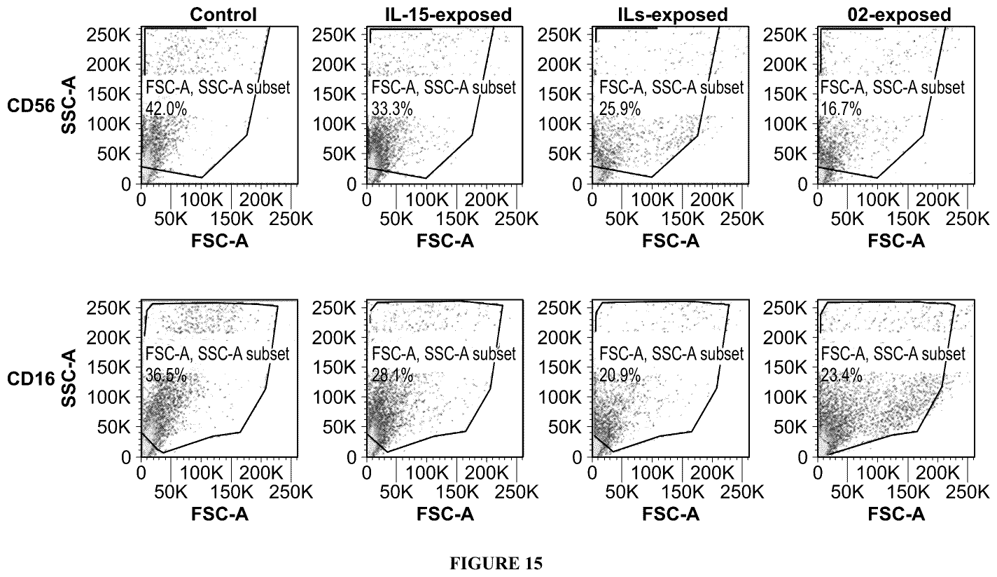

These NK cells were further cultured in the NK differentiation media to achieve functionally mature CD56^{bright} CD16^{bright} NK cells (FIG. 3B, panel (i)). The CD16-expressing NK cells have shown a higher ability to kill cancer cells. The immunofluorescence results (FIG. 3B, panel (ii)) indicated the positive expression of CD56 and CD16 markers, which was followed by flow cytometric quantification of cells that express CD56 (53.1%) and CD16 (45.7%) (FIG. 3B, panel (iii) and FIG. 11A). Markers such as CD7, CD5, GATA3, and CD3D (FIGS. 14A and 14B) were also positive, which indicates the presence of some lymphoid progenitors or NKT populations. Cytometric data were also obtained for other general NK markers (FIG. 3B, panel (iii))-NKG2D (3.49%), NKp30 (33.7%), NKp46 (3.45%), TRAIL (14.3%), and FasL (15.6%). Together, these results revealed the maturation of CD56^{bright} CD16^{-ve} cells toward CD56^{bright} CD16^{bright} subset of NK cells.

Exposure to ATRT Cells and IL-15 Upregulated Activation Markers in and Cytokine Secretion by CD56^{bright} CD16^{bright} iPSC-NK Cells—Activation Using ATRT Cells.

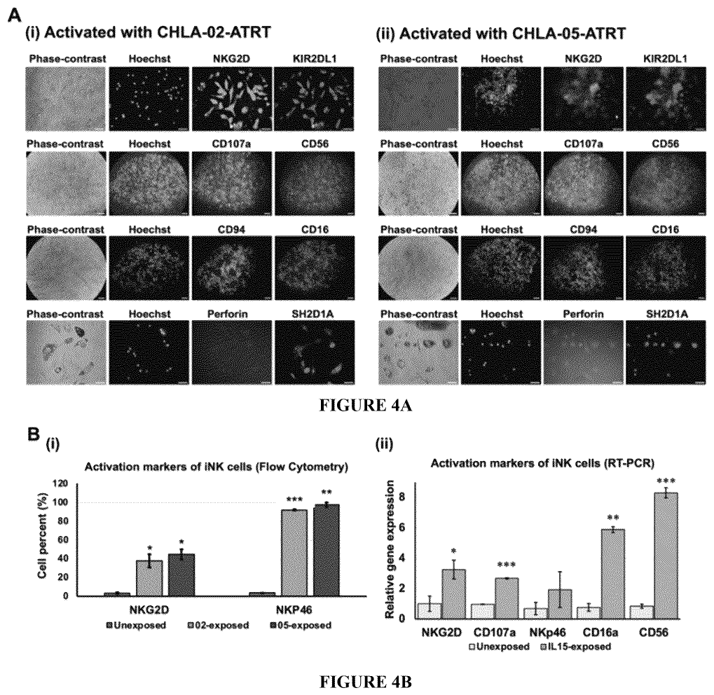

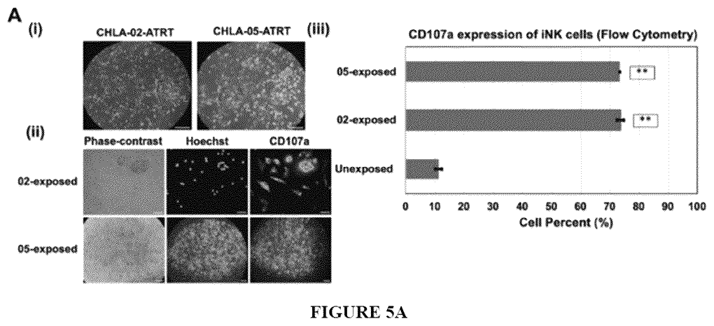

The iPSC-NK cells at maturation stage 1 (CD56^{bright} CD16^{bright}) were grown with CHLA-02-ATRT and CHLA-05-ATRT cells and the expression of various activation markers was measured, such as NKG2D and SH2D1A (Src homology 2 domain-containing protein 1A), inhibitory markers including KIR2DL1 and CD94, cytotoxicity marker CD107a and perforin, and NK cell-specific makers CD56 and CD16. SH2D1A plays an important role in the activation of immune cells, including NK cells. Moreover, CD94 and NKG2D work together to maintain the survival of NK cells. The results as shown in FIG. 4A showed positive expression of these markers, indicating the successful activation of NK cells. The expression of NKp46 and NKG2D activation markers in iPSC-NK cells was significantly upregulated after coculture with ATRT cells as measured via flow cytometry (FIG. 4B, panel (i) and FIGS. 11B and 11C). Cytometric data also confirmed the expression of the NKp30 costimulatory receptor on these activated cells (FIGS. 11B and 11C).

### Activation Using IL-15

The role of interleukin (IL)-15 in the activation of human iPSC-NK cells was also investigated. The cells were exposed to IL-15 for 24 h, and NK cell-specific and activation markers were studied at mRNA levels. The results indicated that the expression of CD16a and CD56 mRNA was 6- and 8-fold higher upon exposure to IL-15 (FIG. 4B, panel (ii)). Moreover, the activation markers NKG2D, CD107a, and NKp46 were also upregulated by 3-, 2.5-, and 2-fold, respectively (FIG. 4B, panel (ii)).

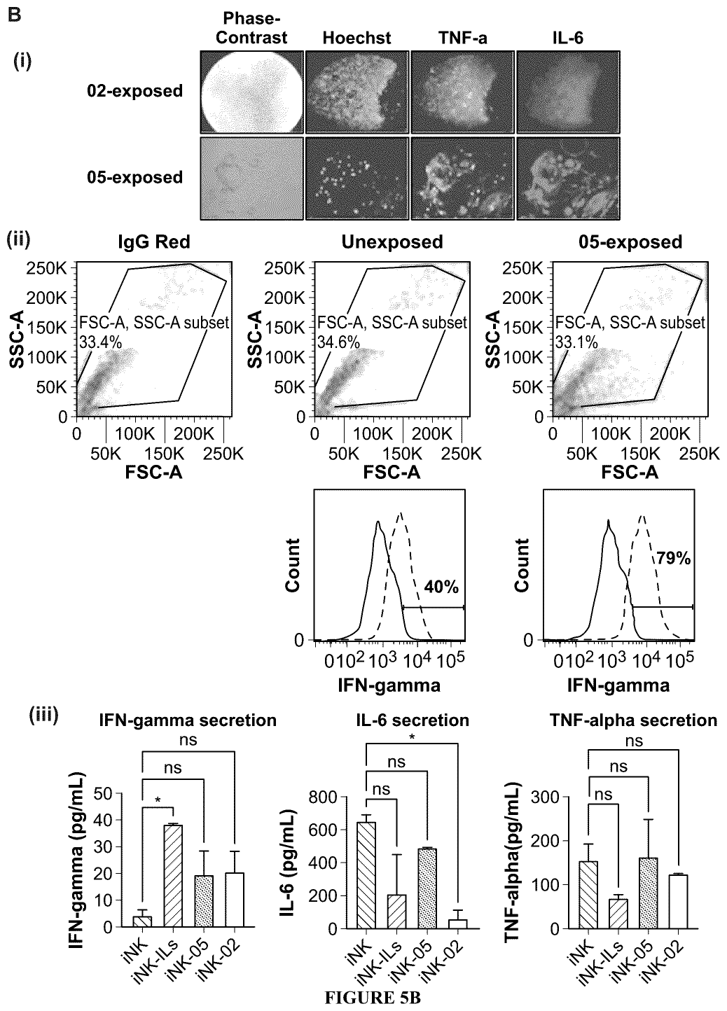

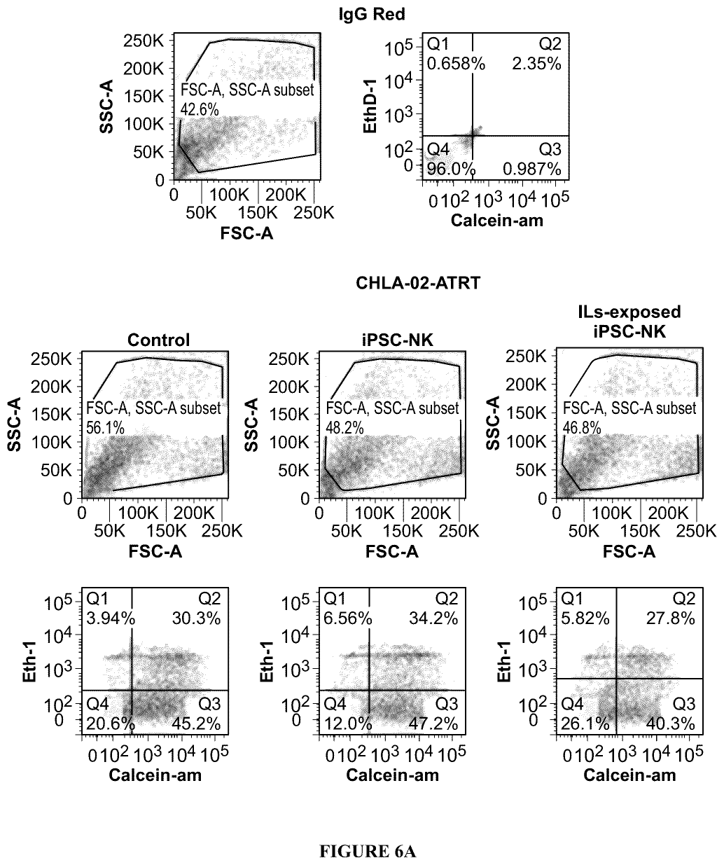

The cytotoxic activity of NK cells can be measured by the CD107a/LAMP1 (lysosomal-associated membrane protein-1) marker on NK cell surface. This marker is associated with the release of lytic substances called perforin and granzymes from granules—a process called degranulation. This process activates the killing ability of NK cells, and CD107a/LAMP1 shows higher expression in activated NK cells during degranulation. Immunofluorescent imaging of iPSC-NK cells after coculture with CHLA-02-ATRT and CHLA-05-ATRT cells (FIG. 5A, panel (i)) showed positive expression of the CD107a marker (FIG. 5A, panel (ii)). The level of CD107a expression was compared before and after NK activation using flow cytometry. The results showed high expression (>70%) of CD107a/LAMP1 in the activated NK cells (FIG. 5A, panel (iii)), implying CD56^{bright} CD16^{bright} iPSC-NK cells can attack target ATRT cells through a degranulation mechanism.

Similarly, when NK cells get activated, they release cytokines, especially interferon (IFN)-γ. IFN-γ production potentially increases the Fas-ligand-based cytotoxicity of NK cells. The expression of IL-6 and tumor necrosis factor (TNF)-α (FIG. 5B, panel (i)) was positive in ATRT-activated iPSC-NK cells. Flow cytometry results showed that the expression of IFN-γ increased significantly from 40% to 79% after exposure to tumor cells, showing one of the functional antitumor mechanisms of CD56^{bright} CD16^{bright} iPSC-NK cells (FIG. 5B, panel (ii)). The quantification of cytokine release revealed an overall increase in IFN-γ secretion by activated NK cells compared to normal iPSC-NK; however, only interleukin-exposed (IL-2, IL-12, IL-15, IL-21, and IL-18) cells manifested a significant difference. Interestingly, IL-6 secretion tended to decrease in activated NK cells, with a noteworthy reduction observed in cells exposed to CHLA-02-ATRT (FIG. 5B, panel (iii)). TNF-α secretion did not change significantly in activated NK cells compared to normal iPSC-NK cells. However, its release was the highest in the CHLA-05-ATRT-activated group compared to other activated groups.

CD56Bright CD16Bright iPSC-NK Cells Showed Functional Cytotoxicity Against ATRT and Other Tumor Cells.

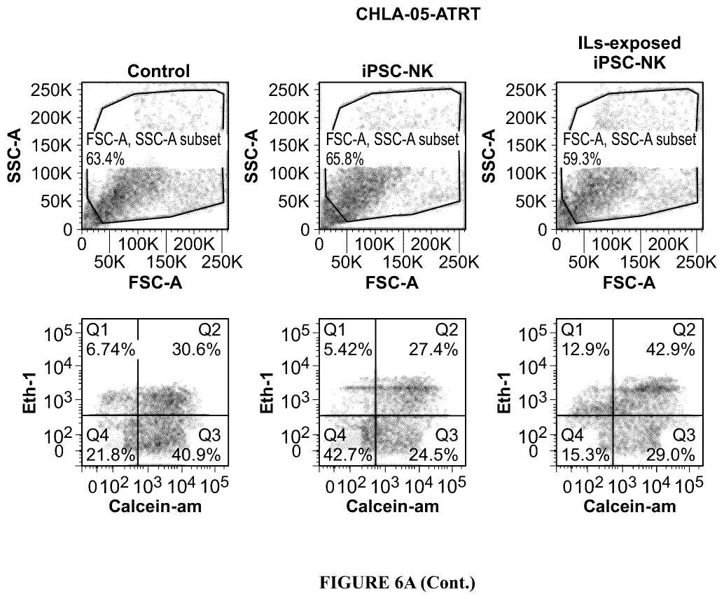

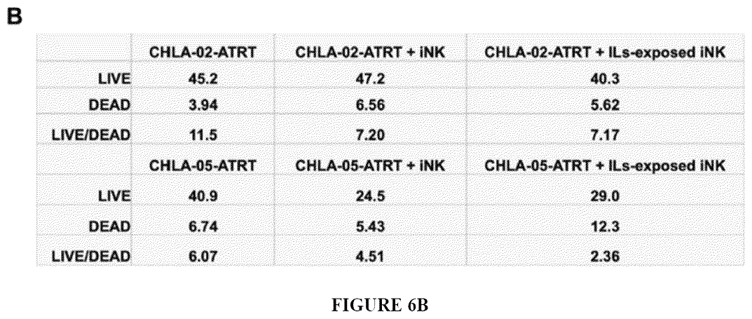

To test the anti-tumor activity, iPSC-NK cells were cocultured with ATRT cells at various effector-to-target ratios. Additionally, the effect of cytokine priming on cytotoxicity was also investigated by exposing iPSC-NK cells to various interleukins. Cytokines such as IL-2, IL-15, and IL-18 have previously been proven to enhance the cytotoxicity of NK cells. IL-2, IL-15, and IL-12 increase the activation and cytotoxicity of NK cells by upregulating the function of TRAIL (TNF-related apoptosis-inducing ligand) and fas-ligand. IL-2 and IL-12 have been shown to work together to induce cytotoxicity of NK cells by increasing their IFN-γ secretion. Moreover, the combined exposure of IL-21, IL-15, and IL-18 enhanced the production of IFN-γ in NK cells. The present disclosure tested the collective role of cytokines IL-2, IL-12, IL-15, IL-21, and IL-18 toward cytotoxicity of iPSC-NK cells against CHLA-02-ATRT and CHLA-05-ATRT. According to the results of live/dead flow cytometry (FIG. 6), the ratio of live to dead ATRT cells was 7.2 for CHLA-02-ATRT and 4.51 for CHLA-05-ATRT in coculture with IL-exposed iPSC-NK cells. The ratio was 7.17 for CHLA-02-ATRT and 2.36 for CHLA-05-ATRT in coculture with IL-unexposed iPSC-NK cells. These ratios were compared with the values of ATRT cells only, which were 11.5 for CHLA-02-ATRT and 6.07 for CHLA-05-ATRT. Hence, the ratio of live to dead ATRT cells was lower in coculture with IL-exposed iPSC-NK cells compared to IL-unexposed iPSC-NK cells.

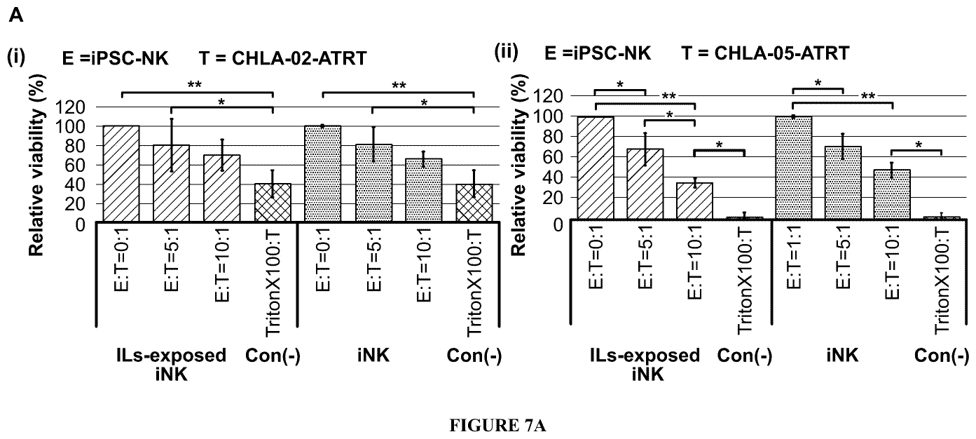

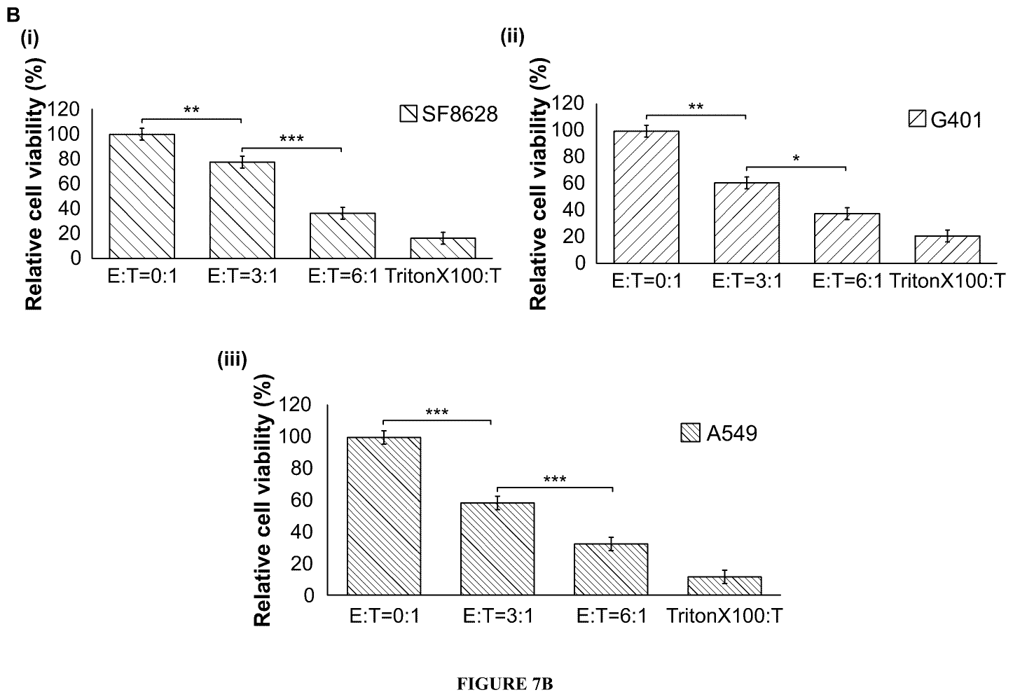

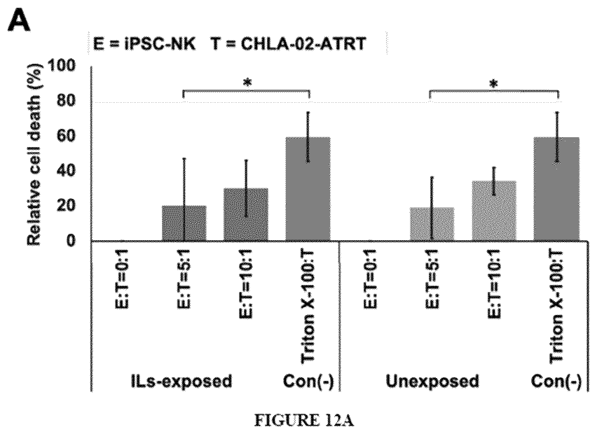

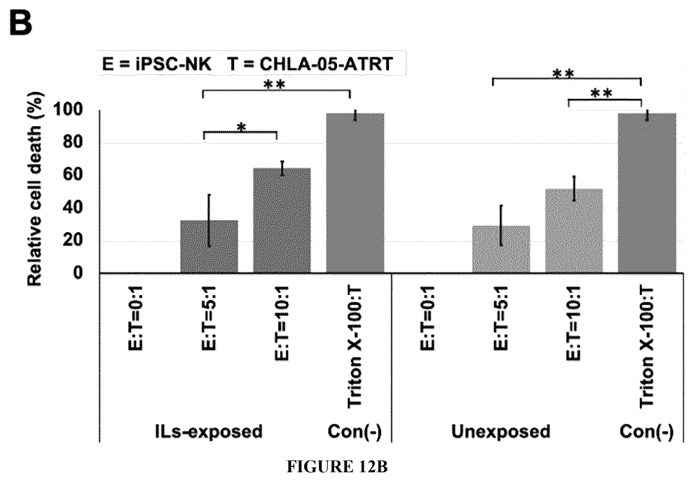

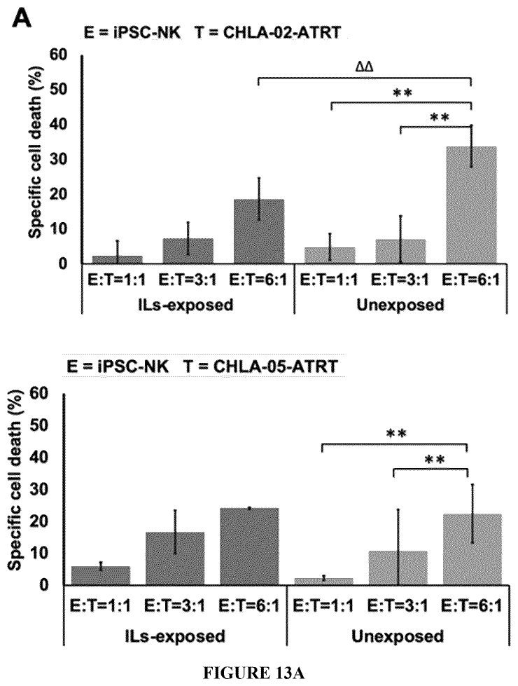

Additionally, the killing of ATRT cells by human iPSC-NK cells was investigated at multiple E:T ratios using MTT assay and Calcein-AM cytotoxicity assay. MTT assay was performed to measure the relative cell viability of ATRT cells by the action of both IL-exposed and IL-unexposed iPSC-NK cells (FIG. 7A). The E:T ratios chosen to calculate the percentage of relative cell viability were 0:1 (negative control), 5:1, and 10:1, along with a positive control (4% Triton X-100). According to the results, a ratio-dependent decrease in cell viability of ATRT cells was observed. At the highest ratio (10:1), the percentage viability of ATRT cells by IL-exposed iPSC-NK cells was 70% for CHLA-02-ATRT and 36% for CHLA-05-ATRT. The percentage of cell viability was 66% (CHLA-02-ATRT) and 48% (CHLA-05-ATRT) for IL-unexposed iPSC-NK cells. At all ratios, the percentage viability of CHLA-05-ATRT was higher compared to CHLA-02-ATRT cells, indicating higher cytotoxic effects of iPSC-NK cells toward CHLA-05-ATRT cells. These results, along with relative cell death results (FIGS. 12A and 12B), were consistent with the cytotoxic assay data in FIG. 7.

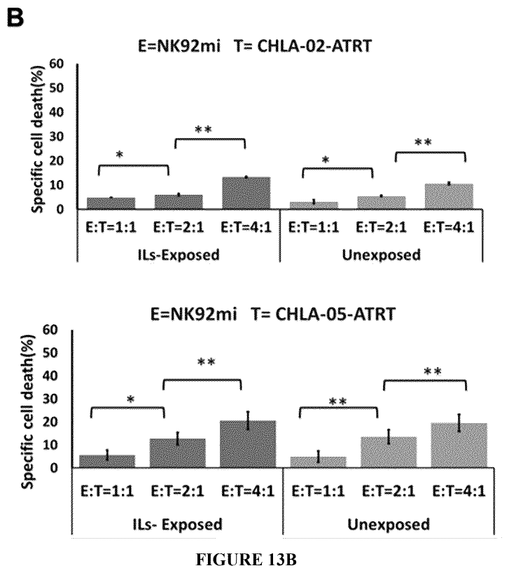

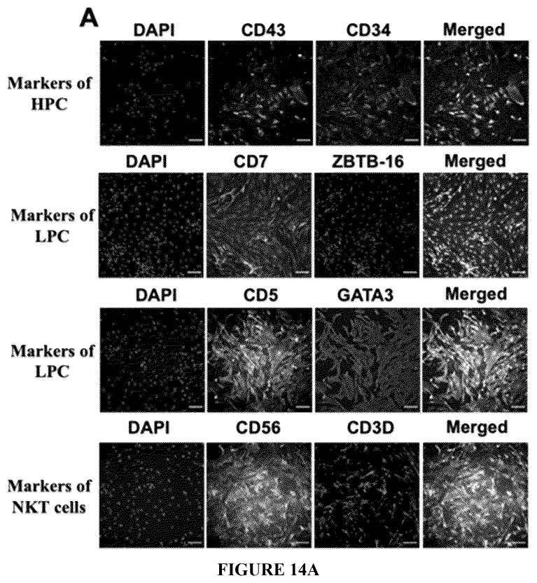

To compare the cytotoxic potential of iPSC-NK cells with other types of NK cells, NK92mi cells (ATCC, Cat #CRL-2408) were studied. NK92mi cells are derived from the peripheral blood of a non-Hodgkin's lymphoma patient and have displayed cytolytic activity against renal cell cancer, multiple myeloma, and Hodgkin lymphoma. Therefore, these NK cells were used for the comparative cytotoxic analysis of iPSC-NK cells. The antitumor activity of NK92mi cells was tested in both ATRT cells-CHLA-02-ATRT and CHLA-05-ATRT (FIGS. 13A and 13B). The percentages of the specific lysis of ATRT cells were determined using a Calcein-AM-based cytotoxicity assay. The anti-tumor activity of iPSC-NK cells and NK92mi cells at various ratios was measured. For CHLA-02-ATRT, the percentage lysis by IL-exposed and unexposed iPSC-NK cells at the E:T ratio of 6:1 was 18% and 33%, respectively (FIG. 13A). While the lysis of CHLA-02-ATRT by NK92mi cells at the E:T ratio of 4:1 was 11% and 13% percentage (FIG. 13B). For CHLA-05-ATRT, the percentage lysis by IL-exposed and unexposed iPSC-NK cells at the same E:T ratio was 24% and 22%, respectively. Whereas the lysis of CHLA-05-ATRT by NK92mi cells at the E:T ratio of 4:1 was 20% and 19%, respectively. The specific cell lysis data indicated comparable cytolytic activity of both iPSC-NK and NK92mi cells at these ratios.

The anti-tumor activity of iPSC-NK cells was also tested in other tumor cells, i.e., SF8628 (DIPG), G401 and A549. Diffuse intrinsic pontine glioma (DIPG) is an aggressive tumor originates in the ventral pons of the brain stem. SF8628 is a DIPG cell line derived from H3·3K27 M DIPG patient. Similarly, G401 cells are also malignant rhabdoid kidney tumor cells derived from a male child. Along with these two pediatric cells, A549 (lung cancer) cells were used as targets to test the cytotoxic efficacy of iPSC-NK cells (FIG. 7B). The iPSC-NK cells were cocultured with each target at three ratios (E:T=0:1, 3:1, and 6:1). The results indicated effective lysis of these tumor cells by iPSC-NK cells at both 3:1 and 6:1 ratio. At 3:1, A549 cells (58%) had the lowest viability compared to SF8628 (78%) and G401 (61%) (FIG. 7B), showing a greater sensitivity of the lung cancer cells toward iPSC-NK cells. At 6:1, the iPSC-NK cells were showing a similar cytolytic activity. However, at this ratio, SF8628 (36%) and G401 (39%) showed comparable viability (FIG. 7B, panels (i) and (ii)). The iPSC-NK cells were added in all cocultures at three ratios (E:T=0:1, 3:1, and 6:1). The results indicated effective lysis of these tumor cells by iPSC-NK cells at these ratios.

Overall, the ATRT cells were the most aggressive compared to all studied pediatric and non-pediatric tumor cells; however, iPSC-NK cells were able to effectively lyse CHLA-05-ATRT cells. CHLA-02-ATRT cells displayed relatively more resistance to iPSC-NK cells. Among all target cells, the lung tumor cells (A549) were the most sensitive to the iPSC-NK cells. The rhabdoid tumor cells (G401) and glioma cells (SF8628), despite their malignant nature, were also sensitive to the cytotoxicity of iPSC-NK cells.

Further Maturation of iPSC-NK Cells into CD56-Ve CD16Bright Phenotype Abated Cytotoxic Potential.

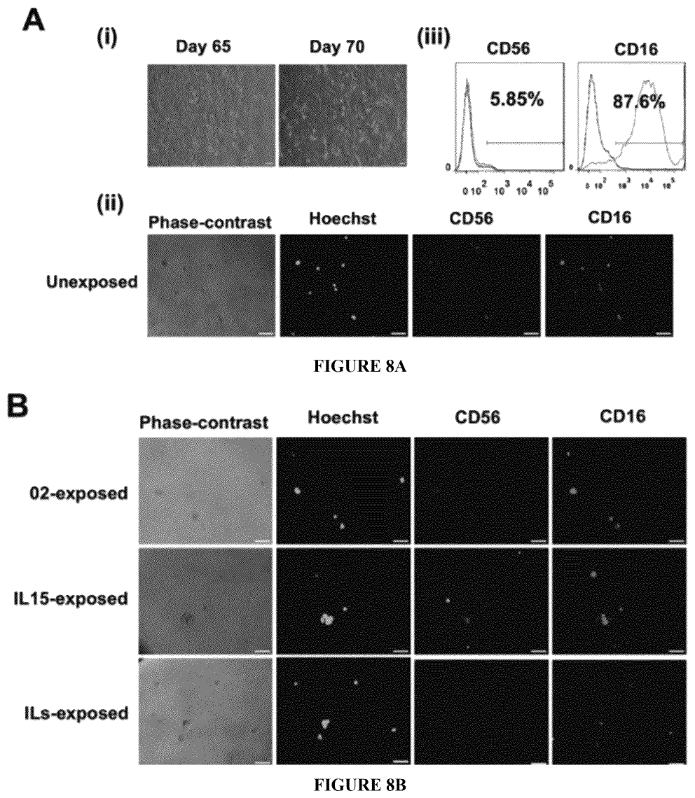

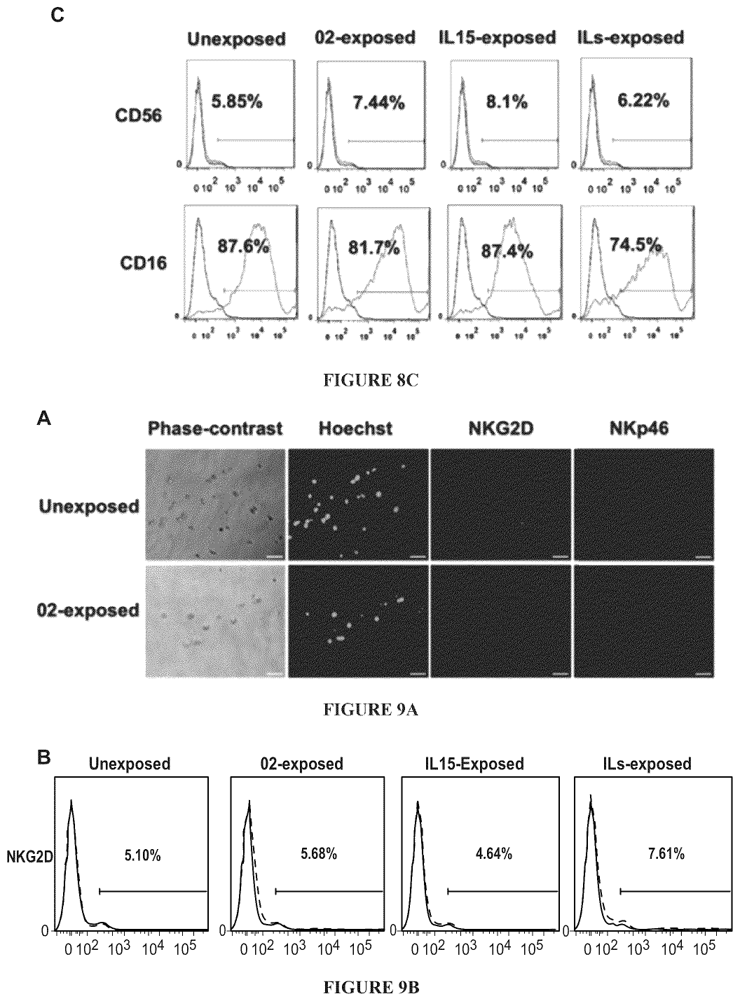

Human iPSC-NK cells were further matured till day 75 (FIG. 8A, panel (i)), and the expression of CD56 and CD16 markers was measured. Negative expression of CD56 and positive expression of CD16 marker was observed (FIG. 8A, panel (ii)). Additionally, the quantitative results showed that the percentage of CD16⁺ cells and CD56⁺ cells was around 5% and 87%, respectively (FIG. 8A, panel (iii)). These cells were exposed to IL-15, the combination of ILs (IL-2, IL-12, IL-15, IL-18, and IL-21), and CHLA-02-ATRT cell to reveal the effects of the exposures on the expression of phenotypic markers CD56 and CD16. The results indicated that the exposures did not have any significant effect on the phenotype of iPSC-NK cells (FIGS. 8B and 8C).

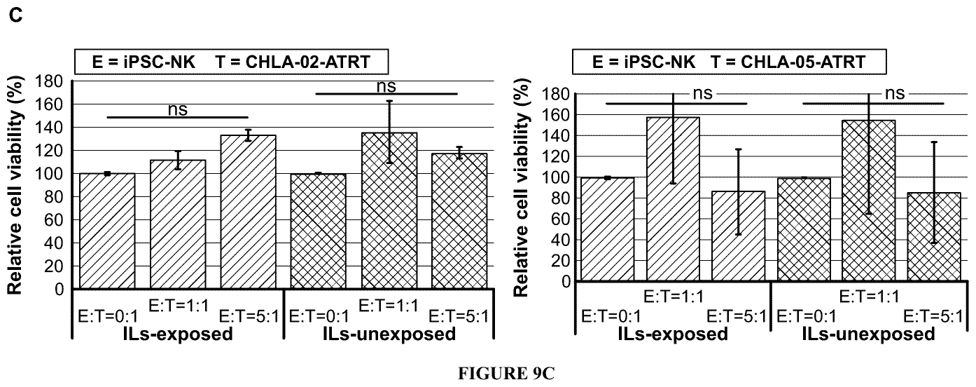

To assess the functionality of CD56^{-ve} CD16^{bright} iPSC-NK cells as effectors, the expression of various cell surface activation markers was determined, and the cytotoxicity of iPSC-NK cells was tested. The results indicated a negative expression of NKG2D and NKp46 activation markers (FIGS. 9A and 9B). This showed the loss of cytotoxic properties of NK cells associated with NKG2D and NKp46 in CD56^{-ve} CD16^{bright} cells. Additionally, the cytotoxicity test showed no change in cell viability of ATRT cells upon exposure to CD56^{-ve} CD16^{bright} iPSC-NK cells (FIG. 9C). The lack of functional properties of these iPSC-NK cells showed possible senescence of the cells.

### DISCUSSION

The human iPSC-derived NK cells provide off-the-shelf immunotherapy and display effective antitumor activity in many in vitro and in vivo models. The present disclosure evaluated various maturational stages of human NK cells during their feeder-free differentiation and maturation from human iPSCs. The demonstration of iPSCs differentiating into effector NK cells in feeder-free culture conditions forms a basis for a non-immunogenic and efficacious immunotherapeutic modality.

The first stage of differentiation is the hematopoietic progenitor stage, characterized based on the high expression of CD34 and CD43 markers. The second stage is characterized by the ability of the progenitors to achieve lymphoid (CD7^{bright}) and innate immune cell lineage (ZBTB-16^{bright}). ZBTB-16 is also known as promyelocytic leukemia zinc finger (PLZF), which enables lymphoid progenitors to attain innate immune cell properties and was highly positive in innate immune cell progenitors. In the third stage, the lymphoid progenitors committed toward CD56^{bright} CD16^{-ve} phenotype of NK cells. This phenotype is known to have a longer telomere than the CD56^{-ve} subset. Therefore, CD56^{bright} CD16^{-ve} cells are deemed to be an immature subset of NK cells that lack cytotoxic function and exhibit immune regulatory properties.

The maturation of NK cells resulted in CD56^{bright} CD16^{bright} phenotype (stage IV), which is known to be a functionally active and cytotoxic subtype. CD16, also known as Fc receptor γRIII, is associated with antibody-dependent cell-mediated cytotoxicity (ADCC). Therefore, the CD16^{bright} NK cell subtype can effectively attack cancer cells by engaging their Fc receptor (CD16) to bind the Fc portion of the mediator antibody that recognizes tumor-associated antigens. The CD16^{bright} iPSC-NK cells are cytotoxic against different tumor cells, including human leukemia K562 cells. Additionally, CD56^{bright} CD16^{bright} NK cells from the peripheral blood of donors displayed cytotoxic potential in patients with refractory acute myeloid leukemia. Thus, obtaining CD56^{bright} CD16^{bright} NK cells from iPSCs for eliminating malignant cells is an avenue in cancer management.

To elucidate the response of CD56^{bright} CD16^{bright} (stage IV) iPSC-NK cells to IL exposure, cells were exposed to IL-15 for 24 h. IL-15 was the choice of interleukin because it has previously been found to control tumor growth by improving the killing potential of NK cells and the proliferation of NK cells both in vitro and in vivo. The exposure resulted in an upregulation of activation, cytolytic, and NK cell-specific makers in iPSC-NK cells, validating the attainment of effector role by these cells.

The CD56^{bright} CD16^{bright} NK cells showed higher expression of activation markers and release of cytokines IFN-γ and TNF-α upon exposure to ATRT cell lines. Previous findings show that the release of IFN-γ improves the cytolytic potential of NK cells and reduces tumor metastasis. Beyond these mechanisms, NK cells release lytic granules carrying perforin and granzyme B to attack their target, CD107a, which is a marker for the degranulation of NK cells, was also seen to be highly expressed by activated iPSC-NK cells. These findings cumulatively show the functional maturation of the derived NK cells and their capability to employ a repertoire of cytotoxic mechanisms post-activation.

The efficacy of CD56^{bright} CD16^{bright} iPSC-NK cells against the ATRT cell lines was tested. Cell viability and cell lysis data showed a higher killing of CHLA-05-ATRT compared to that of CHLA-02-ATRT cells by human iPSC-NK cells. The observed difference in the killing efficiency may be partly explained by varied immunogenicity and sensitivity to apoptosis signals across cancer cells. However, all results showed the ratio-dependent cytotoxicity of human iPSC-NK cells toward both ATRT cell lines. The iPSC-NK cells also exhibited cytolytic functionality toward other aggressive tumor cells, such as glioma (SF8628) and kidney rhabdoid (G401) cells, as well as lung cancer (A549) cells. The anti-tumor activity of iPSC-NK cells among diverse tumor types in vitro indicates their potentiality in cell-based anticancer immunotherapy.

The CD56^{bright} CD16^{bright} phenotype of NK cells was allowed to mature further into the CD56^{-ve} CD16^{bright} subtype. Cells of this subtype lacked the expression of NKG2D and NKp46 markers, implying their functional impairment. The CD56^{-ve} CD16^{bright} NK cells also did not show any significant cytotoxicity when exposed to ATRT cells. Such dysfunctionality of CD56^{-ve} CD16^{bright} NK cells may result from cell senescence and physiological aging.

The present disclosure provides a novel perspective of iPSC-NK cell derivation by identifying various phenotypes of NK cells at each stage of their development and maturation. Moreover, the cytotoxic potential of the identified mature phenotypes of NK cells was tested against rare pediatric tumors such as ATRT cells, which has not yet been reported. The derived NK cells displayed cytotoxic properties at stage IV of their differentiation, but a decline in those properties was observed upon further maturation.

Although there is no absolute cure for pediatric brain tumors, improved survival without cytokine release syndrome has been achieved with NK cell therapies for glioma, medulloblastoma, and high-risk malignant brain tumors. Herein, the iPSC-NK cells showed cytotoxic efficacy toward various malignant tumor cells, including rare pediatric cancer cell lines. These in vitro cytotoxicity studies, however, can only provide a foundational understanding of their short-term functional properties. Investigating their post-infusion persistence, expansion capacity, and long-term efficacy in complex in vivo environments is required to advance the knowledge about their immune properties for therapeutic use, presenting a translational future research direction.

In summary, the present disclosure demonstrates the derivation of common NK cell subtypes from iPSCs, including the CD56^{bright} CD16^{bright} phenotype with in vitro effector function. The characterization of their cytotoxic properties, antitumor activity, and aging-induced functional decrement serves as the basis for testing the in vivo persistence and efficacy of NK cells.

The present disclosure describes a feeder-free differentiation of NK cells from iPSCs. Various maturational subtypes of iPSC-NK cells were characterized and their phenotypic and functional properties were studied. Mature iPSC-NK cells of CD56^{bright} CD16^{bright} phenotype expressed activation markers in response to interleukin stimuli and produced cytokines upon exposure to malignant brain rhabdoid tumor cells. Moreover, CD56^{bright} CD16^{bright} iPSC-NK cells demonstrated functional cytotoxicity by killing malignant cells. However, further maturation of the cells yielded a dysfunctional CD56^{-ve} CD16^{bright} subtype. The results show that iPSC-NK cells with effector properties can form the basis of an effective therapeutic strategy against aggressive pediatric brain tumors.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### TABLES

TABLE 1
A brief description of NK cell phenotype,
activation, and cytotoxicity markers.

| Markers | Specificity |
|---|---|
| CD56 | NK cell-specific marker |
| CD34, CD43 | Hematopoietic progenitor markers |
| CD45 | Common hematopoietic cell marker |
| ZBTB16/PLZF | Innate cell immunity |
| CD3 | T cell co-receptor |
| CD16 | Expressed in large granular |
| | lymphocytes |
| CD94 | CD94/NKG2 inhibitory receptors |
| | in developing NK |
| KIR2DL1/CD158a | Killer cell inhibitory receptor |
| | expressed in subset of NK |
| NKG2D | Activating receptors of NK cell |
| Activating receptors | |
| of NK cell | |
| NKP30 | |
| LAMP1/CD107a | LAMP1/CD107a |
| CD107a | Stimulated NK cells |
| IFN-γ | |
| TNF-α | |

CD: Cluster of differentiation, ZBTB: Zinc Finger and BTB Domain; PLZF: Promyelocytic leukemia zinc finger protein; KIR2DL1: Killer-cell immunoglobulin-like receptors; NKG2D: Natural Killer Group 2D; LAMP1: Lysosome-associated membrane glycoprotein 1; IFN-γ: Interferon γ; TNF: Tumor Necrosis Factor.

TABLE 2
Sequences of forward and reverse primers for
RT-PCR.

| | Orien- | | SEQ |
|---|---|---|---|
| Gene | tation | Sequences (5′ to 3′) | ID NO |
| CD16 | F | CAAATCCTTCATCATGTCAGTTC | 1 |
| | | C | |
| | R | GAACCAAGAAATGTTGCGCTTA | 2 |
| CD56 | F | GCAGATGGGAGAGGATGGAAA | 3 |
| | R | TCTGGTTTCCACTCGGAGGAG | 4 |
| NKG2D | F | TCAAGATCTTCCCTCTCTGAGCA | 5 |
| | R | CGAATCCACCCCATCAAATAC | 6 |
| NKp46 | F | ACTAGGCCGGCAGAATCTGAG | 7 |
| | R | CGAGAGGGTGGGTGTGTCATA | 8 |
| CD107a | F | TCACACGTAGGACGCATGAAG | 9 |
| | R | GAAGCGCTCCAGACACTCATC | 10 |

What is claimed is:

1. A method of treating a cancer in a subject in need thereof, the method comprising administering to the subject a cell-based immunotherapy comprising an induced pluripotent stem cell-derived natural killer cell (iPSC-derived NK cell), wherein the iPSC-derived NK cell is prepared by (i) obtaining an induced pluripotent stem cell (iPSC); (ii) culturing the iPSC to differentiate into a hematopoietic progenitor cell; (iii) culturing the hematopoietic progenitor cell to further differentiate into a lymphoid progenitor cell; (iv) culturing the lymphoid progenitor cell to further differentiate into an immature NK cell; and (v) culturing the immature NK cell to further differentiate into the iPSC-derived NK cell, wherein the iPSC-derived NK cell is activated in a feeder cell-free cell culture, and wherein the iPSC-derived NK cell comprises comparable cytotoxicity relative to a control NK cell.

2. The method of claim 1, wherein the hematopoietic progenitor cell is a CD34⁺, CD43⁺ cell.

3. The method of claim 1, wherein the lymphoid progenitor cell is a CD7⁺, ZBTB16⁺ cell.

4. The method of claim 1, wherein the immature NK cell is a CD56⁺, CD16⁻ NK cell.

5. The method of claim 1, wherein the iPSC-derived NK cell is a CD56⁺, CD16⁺ NK cell.

6. The method of claim 1, wherein the iPSC-derived NK cell is further activated in the presence of interleukin-15 (IL-15).

7. The method of claim 1, wherein the iPSC-derived NK cell comprises increased expression of NKG2D, CD107a, NKp46, CD16a, CD56, or a combination thereof relative to control.

8. The method of claim 1, wherein the iPSC-derived NK comprises an increased release of interferon-gamma (IFN-γ) and a decreased release of IL-6 and tumor necrosis factor-alpha (TNF-α), or a combination thereof relative to a control.

9. The method of claim 1, wherein the cancer comprises atypical teratoid rhabdoid tumor (ATRT), diffuse intrinsic pontine glioma (DIPG), lung cancer, non-Hodgkin's lymphoma, renal cell cancer, multiple myeloma, or Hodgkin's lymphoma.

10. The method of claim 1, wherein the iPSC-derived NK cell causes degranulation in a cancer cell.

11. A method of differentiating a human induced pluripotent stem cell (iPSC) into a mature natural killer (NK) cell in a feeder cell-free cell culture, the method comprising:
a. obtaining a human induced pluripotent stem cell (iPSC);
b. culturing the human iPSCs to differentiate into a hematopoietic progenitor cell;
c. culturing the hematopoietic progenitor cell to further differentiate into a lymphoid progenitor cell;
d. culturing the lymphoid progenitor cell to further differentiate into an immature NK cell; and
e. culturing the immature NK cell to further differentiate into a mature NK cell.

12. The method of claim 11, wherein the hematopoietic progenitor cell is a CD34⁺, CD43⁺ cell.

13. The method of claim 11, wherein the lymphoid progenitor cell is a CD7⁺, ZBTB16⁺ cell.

14. The method of claim 11, wherein the immature NK cell is a CD56⁺, CD16⁻ NK cell.

15. The method of claim 11, wherein the mature NK cell is a CD56⁺, CD16⁺ NK cell.

16. The method of claim 11, wherein the human-derived iPSC NK cell is activated in the presence of interleukin-15 (IL-15).

17. The method of claim 11, wherein the mature NK cell comprises increased expression of NKG2D, CD107a, NKp46, CD16a, CD56, or a combination thereof relative to control.

18. The method of claim 11, wherein the mature NK comprises an increased release of interferon-gamma (IFN-γ) and a decreased release of IL-6 and tumor necrosis factor-alpha (TNF-α), or a combination thereof relative to a control.

19. The method of claim 11, wherein the method comprises treating a cancer selected from atypical teratoid rhabdoid tumor (ATRT), diffuse intrinsic pontine glioma (DIPG), lung cancer, non-Hodgkin's lymphoma, renal cell cancer, multiple myeloma, or Hodgkin's lymphoma.

20. The method of claim 11, wherein the iPSC-derived NK cell causes degranulation in a cancer cell.
